(19) United States
(12) Patent Application Publication
(10) Pub. No.: US 2024/0270676 A1
(43) Pub. Date: Aug. 15, 2024

(54) NONANIC ACID ESTERS

(71) Applicant: Evonik Operations GmbH, Essen (DE)

(72) Inventors: Stefan Julian Liebig, Duesseldorf (DE); Jan Marian von Hof, Bochum (DE); Tobias Karl Hubert Müller, Limburg an der Lahn (DE); Maxim Yavorsky, Witten (DE); Sunay Karacocuk, Herne (DE); Alexandra Trambitas, Alzenau (DE); Dominik Schuch, Duesseldorf (DE)

(73) Assignee: Evonik Operations GmbH, Essen (DE)

(21) Appl. No.: 18/570,018

(22) PCT Filed: May 30, 2022
(86) PCT No.: PCT/EP2022/064529

§ 371 Date: Dec. 13, 2023

(30) Foreign Application Priority Data
Jun. 18, 2021 (EP) 21180241.8

(51) Int. Cl. C07C 69/33; C12P 7/6454

(52) U.S. Cl. CPC C07C 69/33; C12P 7/6454

(57) ABSTRACT

n-Nonanoic esters of xylitol, sorbitol or erythritol are made, especially for cosmetic or household care compositions, and a process for their preparation is developed.

### FIELD OF THE INVENTION

The invention provides n-nonanoic esters of xylitol, sorbitol or erythritol, a process for preparation thereof, and for the use thereof in cosmetic compositions in particular.

### PRIOR ART

### Nonanoic Acid/Pelargonic Acid

n-Nonanoic acid (pelargonic acid, CAS 112-05-0) can be obtained by oxidation of n-nonanal of petrochemical origin (“Carboxylic Acids, Aliphatic,” in: Ullmann's Encyclopedia of Industrial Chemistry 2014). Alternatively, n-nonanoic acid can be obtained by ozonolysis of ω-9-fatty acids, for example oleic acid and erucic acid, or esters thereof. However, ozonolysis is a process having high energy demand and specific process requirements, for example the use of an ozone generator. Moreover, the ω-9-fatty acids used have often been obtained from tropical oils, for example palm oil, palm kernel oil and coconut oil. Much more sustainable processes for preparing n-nonanoic acid are based on hydrogen peroxide (Soutelo-Maria et al. in Catalysts 2018, 8, 464), particularly processes as, for example, in U.S. Pat. Nos. 9,272,975, 8,846,962, 8,222,438, WO2007039481 and WO2011080296, if they are also conducted proceeding from ω-9-fatty acids or esters thereof that have not been obtained from tropical oils.

Esters of Xylitol and n-Nonanoic Acid

Savelli et al. in International Journal of Pharmaceutics 1999, 182, 221-23 describe the regioselective synthesis of the pure stereoisomer 1-O-nonanoyl-D,L-xylitol and its properties as amphiphile (water solubility, critical micelle concentration (CMC), surface tension, formation of lyotropic liquid crystals, HLB value). A disadvantage of the process described in the prior art is the three-stage synthesis in the presence of organic solvents with use of isopropylidene protecting groups. Nonanoyl chloride is used here for the acylation, which is likewise a disadvantage. Similar studies, for example the determination of the transition temperatures, are described for 1-O-nonanoyl-D,L-xylitol obtained by the same synthesis route by Goodby et al. in Liquid Crystals 1997, 22, 367-378 and Douillet et al. in FR 2728257 A1. Dahlhoff et al. in Zeitschrift fuer Naturforschung, B: Chemical Sciences 1996, 51, 1229-1234, for synthesis of 1-O-nonanoyl-D,L-xylitol, chose boranediyl protecting groups that were even installed in carcinogenic benzene as solvent. Here too, the properties of the pure 1-O-nonanoyl-D,L-xylitol stereoisomer are examined in liquid crystal form.

Esters of Sorbitol and n-Nonanoic Acid

EP879872 discloses fully esterified sorbitol hexanonanoate as a constituent of lubricant oil compositions.

### Other Prior Art

KR101939851B1 describes esters of dehydrated xylitol and the use of these carboxylic esters of anhydroxylitol as rheological additive/viscosity regulator in an emulsion. One disadvantage of the anhydroxylitol carboxylates described in the prior art is their reduced hydrophilicity. A further disadvantage of the anhydroxylitol carboxylates described in the prior art is their dark colour. A further disadvantage of such anhydroxylitol carboxylates is the lack of thickening performance in aqueous surfactant systems.

DE102009001748A describes sorbitan esters obtained from the solvent-free reaction of 1 mol of sorbitol (also called glucitol) with 1.55 mol of caprylic acid, and the use of the sorbitan esters thus obtained as thickener for aqueous surfactant systems. It is a disadvantage of the process that, under the reaction conditions described, the sorbitol is dehydrated virtually completely, but at least partially, and forms what is called sorbitan (a product mixture). Moreover, discoloured and odorous products are obtained, which do not meet quality standards for cosmetic applications without additional bleaching or treatment with activated carbon.

The problem addressed by the invention was that of providing n-nonanoic esters that are able to overcome at least one disadvantage of the prior art.

### DESCRIPTION OF THE INVENTION

It has been found that, surprisingly, the n-nonanoic esters described hereinafter and the process described hereinafter are capable of solving the problem addressed by the invention.

It is an advantage of the present invention that the n-nonanoic esters according to the invention are excellent thickeners for aqueous surfactant systems compared to the prior art.

It is a further advantage that the n-nonanoic esters according to the invention also have excellent colour and very good odour compared to the prior art.

It is an advantage of the process of the present invention that only a very low level of degradation products of the sugars or sugar alcohols used or esters of the degradation products are obtained as reaction products.

It is an advantage of the present invention that the process according to the invention can be performed in the absence of a solvent.

It is an advantage of the present invention that the process according to the invention can be performed in one reaction step.

It is an advantage of the present invention that the process according to the invention can be performed without protecting group chemistry.

It is a further advantage of the present invention that the n-nonanoic esters can be obtained in a homogeneous reaction mixture, so that no additional process steps such as extraction, crystallization, filtration or distillation, for example, are required.

It is an advantage of the present invention that the process can be performed at elevated temperatures. This leads to better miscibility of the co-reactants, while the recyclability of the enzyme used is surprisingly high.

It is a further advantage of the present invention that the n-nonanoic esters obtained can be incorporated very readily into formulations, particularly into cosmetic formulations and household care formulations.

The present invention therefore provides an n-nonanoic ester of xylitol, sorbitol or erythritol, characterized in that it takes the form of a mixture in which at least two of the esters differ with regard to at least one esterification position of at least one nonanoyl radical in the xylitol, sorbitol or erythritol, with the proviso, that n-nonanoic esters of erythritol with an average level of esterification of greater than 3.2 are excluded.

The present invention thus describes a mixed composition of structurally different esters.

For instance, a n-nonanoic ester preferred in accordance with the invention is characterized in that it comprises at least two regioisomers of the mono-n-nonanoic ester.

The expression “that the n-nonanoic ester takes the form of a mixture in which at least two of the esters differ with regard to at least one esterification position of at least one nonanoyl radical in the xylitol, sorbitol or erythritol” is understood to mean that at least two esters of the same sugar alcohol in each case differ from one another.

In the case of an n-nonanoic ester of xylitol, these different esters may be selected, for example, from
1-O-nonanoyl-xylitol, 2-O-nonanoyl-xylitol, 3-O-nonanoyl-xylitol, 4-O-nonanoyl-xylitol, 5-O-nonanoyl-xylitol,
1,2-O-dinonanoyl-xylitol, 1,3-O-dinonanoyl-xylitol, 1,4-O-dinonanoyl-xylitol, 1,5-O-dinonanoyl-xylitol, 2,3-O-dinonanoyl-xylitol, 2,4-O-dinonanoyl-xylitol, 2,5-O-dinonanoyl-xylitol, 3,4-O-dinonanoyl-xylitol, 3,5-O-dinonanoyl-xylitol, 4,5-O-dinonanoyl-xylitol,
1,2,3-O-trinonanoyl-xylitol, 1,2,4-O-trinonanoyl-xylitol, 1,2,5-O-trinonanoyl-xylitol, 1,3,4-O-trinonanoyl-xylitol, 1,3,5-O-trinonanoyl-xylitol, 1,4,5-O-trinonanoyl-xylitol, 2,3,4-O-trinonanoyl-xylitol,
2,3,5-O-trinonanoyl-xylitol, 2,4,5-O-trinonanoyl-xylitol, 3,4,5-O-trinonanoyl-xylitol,
1,2,3,4-O-tetranonanoyl-xylitol, 1,2,3,5-O-tetranonanoyl-xylitol, 1,2,4,5-O-tetranonanoyl-xylitol,
1,3,4,5-O-tetranonanoyl-xylitol, 2,3,4,5-O-tetranonanoyl-xylitol and
1,2,3,4,5-O-pentanonanoyl-xylitol,
   with particular preference for
1-O-nonanoyl-xylitol, 2-O-nonanoyl-xylitol, 3-O-nonanoyl-xylitol, 4-O-nonanoyl-xylitol, 5-O-nonanoyl-xylitol,
1,2-O-dinonanoyl-xylitol, 1,5-O-dinonanoyl-xylitol, 4,5-O-dinonanoyl-xylitol,
1,2,5-O-trinonanoyl-xylitol and 1,4,5-O-trinonanoyl-xylitol.

In the case of an n-nonanoic ester of sorbitol, these different esters may be selected, for example, from
1-O-nonanoyl-sorbitol, 2-O-nonanoyl-sorbitol, 3-O-nonanoyl-sorbitol, 4-O-nonanoyl-sorbitol, 5-O-nonanoyl-sorbitol, 6-O-nonanoyl-sorbitol,
1,2-O-dinonanoyl-sorbitol, 1,3-O-dinonanoyl-sorbitol, 1,4-O-dinonanoyl-sorbitol, 1,5-O-dinonanoyl-sorbitol, 1,6-O-dinonanoyl-sorbitol, 2,3-O-dinonanoyl-sorbitol, 2,4-O-dinonanoyl-sorbitol, 2,5-O-dinonanoyl-sorbitol, 2,6-O-dinonanoyl-sorbitol, 3,4-O-dinonanoyl-sorbitol, 3,5-O-dinonanoyl-sorbitol, 3,6-O-dinonanoyl-sorbitol, 4,5-O-dinonanoyl-sorbitol, 4,6-O-dinonanoyl-sorbitol, 5,6-O-dinonanoyl-sorbitol,
1,2,3-O-trinonanoyl-sorbitol, 1,2,4-O-trinonanoyl-sorbitol, 1,2,5-O-trinonanoyl-sorbitol, 1,2,6-O-trinonanoyl-sorbitol,
1,3,4-O-trinonanoyl-sorbitol, 1,3,5-O-trinonanoyl-sorbitol, 1,3,6-O-trinonanoyl-sorbitol,
1,4,5-O-trinonanoyl-sorbitol, 1,4,6-O-trinonanoyl-sorbitol, 1,5,6-O-trinonanoyl-sorbitol,
2,3,4-O-trinonanoyl-sorbitol, 2,3,5-O-trinonanoyl-sorbitol, 2,3,6-O-trinonanoyl-sorbitol,
2,4,5-O-trinonanoyl-sorbitol, 2,4,6-O-trinonanoyl-sorbitol, 2,5,6-O-trinonanoyl-sorbitol
3,4,5-O-trinonanoyl-sorbitol, 3,4,6-O-trinonanoyl-sorbitol, 3,5,6-O-trinonanoyl-sorbitol,
4,5,6-O-trinonanoyl-sorbitol
1,2,3,4-O-tetranonanoyl-sorbitol, 1,2,3,5-O-tetranonanoyl-sorbitol, 1,2,3,6-O-tetranonanoyl-sorbitol,
1,2,4,5-O-tetranonanoyl-sorbitol, 1,2,4,6-O-tetranonanoyl-sorbitol, 1,2,5,6-O-tetranonanoyl-sorbitol,
1,3,4,5-O-tetranonanoyl-sorbitol, 1,3,4,6-O-tetranonanoyl-sorbitol, 1,3,5,6-O-tetranonanoyl-sorbitol,
1,4,5,6-O-tetranonanoyl-sorbitol,
2,3,4,5-O-tetranonanoyl-sorbitol, 2,3,4,6-O-tetranonanoyl-sorbitol, 2,3,5,6-O-tetranonanoyl-sorbitol,
2,4,5,6-O-tetranonanoyl-sorbitol and
3,4,5,6-O-tetranonanoyl-sorbitol,
   with particular preference for
1-O-nonanoyl-sorbitol, 2-O-nonanoyl-sorbitol, 5-O-nonanoyl-sorbitol, 6-O-nonanoyl-sorbitol,
1,2-O-dinonanoyl-sorbitol, 1,6-O-dinonanoyl-sorbitol, 5,6-O-dinonanoyl-sorbitol,
1,2,3-O-trinonanoyl-sorbitol, 1,2,6-O-trinonanoyl-sorbitol, 1,5,6-O-trinonanoyl-sorbitol,
4,5,6-O-trinonanoyl-sorbitol,
1,2,4,6-O-tetranonanoyl-sorbitol, 1,2,5,6-O-tetranonanoyl-sorbitol,
1,3,4,6-O-tetranonanoyl-sorbitol, 1,3,5,6-O-tetranonanoyl-sorbitol and 1,4,5,6-O-tetranonanoyl-sorbitol.

In the case of an n-nonanoic ester of erythritol, these different esters may be selected, for example, from
1-O-nonanoyl-erythritol, 2-O-nonanoyl-erythritol, 3-O-nonanoyl-erythritol, 4-O-nonanoyl-erythritol,
1,2-O-dinonanoyl-erythritol, 1,3-O-dinonanoyl-erythritol, 1,4-O-dinonanoyl-erythritol,
2,3-O-dinonanoyl-erythritol, 2,4-O-dinonanoyl-erythritol,
3,4-O-dinonanoyl-erythritol,
1,2,3-O-trinonanoyl-erythritol, 1,2,4-O-trinonanoyl-erythritol, 1,3,4-O-trinonanoyl-erythritol, 2,3,4-O-trinonanoyl-erythritol and
1,2,3,4-O-tetranonanoyl-erythritol,
   with particular preference for
1-O-nonanoyl-erythritol, 2-O-nonanoyl-erythritol, 3-O-nonanoyl-erythritol, 4-O-nonanoyl-erythritol,
1,2-O-dinonanoyl-erythritol, 1,3-O-dinonanoyl-erythritol, 1,4-O-dinonanoyl-erythritol,
2,4-O-dinonanoyl-erythritol,
1,2,4-O-trinonanoyl-erythritol, 1,3,4-O-trinonanoyl-erythritol and
1,2,3,4-O-tetranonanoyl-erythritol.

An n-nonanoic ester preferred in accordance with the invention is characterized in that it comprises mono-n-nonanoic ester and di-n-nonanoic ester, and preferably tri-n-nonanoic ester.

Preferably, the mono-n-nonanoic ester present, in this context, has at least two regioisomers.

It is preferable in accordance with the invention that the n-nonanoic ester according to the invention has an average level of esterification of 1.0 to 4.0, preferably of 1.0 to 3.8, more preferably of 1.1 to 2.5, especially preferably of 1.3 to 2.3, with the proviso, that n-nonanoic esters of erythritol with an average level of esterification of greater than 3.2 are excluded.

See below with regard to the determination of the level of esterification of the n-nonanoic ester according to the invention via GC.

An n-nonanoic ester preferred in accordance with the invention is characterized in that it is present in a mixed composition containing less than 25% by weight, preferably from 0.01% by weight to 20% by weight, especially preferably from 0.05% by weight to 10% by weight, of free n-nonanoic acid, where the percentages by weight are based on the sum total of all n-nonanoic esters of xylitol, sorbitol and erythritol and n-nonanoic acid.

The free n-nonanoic acid may be in protonated or neutralized form.

The content of free n-nonanoic acid in the mixed compositions according to the invention containing the n-nonanoic esters is determined by first determining the acid number. This can be used to determine the proportion by weight of n-nonanoic acid via the molar mass thereof. Suitable methods for determining the acid number are especially those according to DGF C-V 2, DIN EN ISO 2114, Ph. Eur. 2.5.1, ISO 3682 and ASTM D 974.

The saponification value is determined by those skilled in the art in accordance with DGF C-V 3 or DIN EN ISO 3681.

An n-nonanoic ester preferred in accordance with the invention is characterized in that it is present in a mixed composition containing 0.05% by weight to 40% by weight, preferably 0.2% by weight to 25% by weight, especially preferably 0.5% by weight to 10% by weight, the most preferably 2.0% by weight to 8.0% by weight, of free xylitol, sorbitol and/or erythritol, where the percentages by weight are based on the sum total of all n-nonanoic esters of xylitol, sorbitol and erythritol and all xylitol, sorbitol and erythritol.

Mixed compositions preferred in accordance with the invention contain the n-nonanoic ester according to the invention in an amount of 40.0% by weight to 99.5% by weight, preferably 50.0% by weight to 98.0% by weight, especially preferably 40.0% by weight to 95.0% by weight, the most preferably 60.0% by weight to 80.0% by weight, where the percentages by weight are based on the overall mixed composition.

The n-nonanoic esters according to the invention have excellent processibility in liquid form, for example for production of formulations for cosmetic applications in particular.

Therefore, mixed compositions that are preferred in accordance with the invention and comprise n-nonanoic esters according to the invention are characterized in that they contain 0.1% by weight to 60% by weight, preferably 1.0% by weight to 50% by weight, even more preferably 5.0% by weight to 40% by weight, especially preferably 10% by weight to 35% by weight, of at least one solvent.

Preferably in accordance with the invention, these solvents are selected from the groups of
a) 1,2-diols, 1,3-diols, 1,4-diols and α,ω-diols, where the aforementioned preferably have 2 to 8 carbon atoms,
b) polyols, especially glycerol, oligoglycerols, for example diglycerol, and polyglycerols,
c) glycerol fatty acid partial esters, oligoglycerol fatty acid partial esters, for example diglycerol fatty acid partial esters, and polyglycerol fatty acid partial esters, and
d) water.

Particularly preferred solvents are selected from propane-1,3-diol, propylene glycol, glycerol and water.

Preference is given in accordance with the invention to an n-nonanoic ester which is characterized in that the complete diester component of the n-nonanoic ester includes from 10% by weight to 50% by weight, preferably from 15% by weight to 45% by weight, especially preferably from 20% by weight to 35% by weight, of regioisomers in which at least one secondary hydroxyl group has been esterified.

The determination of the level of esterification, the determination of the content of different regioisomers, for example in the complete monoester component and in the complete diester component of the n-nonanoic ester according to the invention, and the determination of the content of triester species based on the sum of all n-nonanoic esters according to the invention that are present, and the determination of the content of regioisomers in the complete diester component of the n-nonanoic ester according to the invention in which at least one secondary hydroxyl group has been esterified can be conducted by means of gas chromatography, optionally coupled to mass spectrometry (GC-FID and GC-MS):

First 100 mg of a sample of the appropriate n-nonanoic ester is dissolved in 5 ml in pyridine/dichloromethane (4:1). Then 0.5 ml of N-methyl-N-(trimethylsilyl)trifluoroacetamide (MSTFA) and 0.5 ml of a mixture of pyridine and trimethylsilylimidazole (39:11) are added.

Derivatization is effected at 80° C. for 30 minutes. A sample of the clear solution thus obtained is analysed by means of GC-FID and GC-MS. The parameters of the analysis method are:
Gas chromatograph: Agilent MSD 7890
Column: Agilent SimDist (10 m, 0.32 mm, 0.1 μm),
Flow rate: a constant 3 ml/min of hydrogen (GC-MS: helium)
Temperature 65° C., 10° C./min; 365° C., 15 min, injector 0.1 μl, on-column,
Detector: FID, 370° C./GC-MS Scan 35-650 d

In the GC-FID analysis, the esters present in the sample are separated according to their total chain length. The ratios of the individual ester species to one another are determined via the respective area percentage of the GC-FID peak. The peaks are identified/assigned to the individual ester species via GC-MS, if appropriate also via a comparison of retention time or separately prepared and isolated standards, for example for the mono- and diesters esterified exclusively at primary hydroxyl groups.

This method can likewise be used to detect the content of free protonated and also free neutralized carboxylic acids, since these are likewise derivatized.

The level of esterification is determined via the sum totals of the respective peak areas of all mono-, di-, tri-, tetra-, penta- and hexaesters:

$${a}_{i} = \frac{{A}_{i}}{{\sum }_{i} {A}_{i}}$$

with aᵢ=normalized sum total of the respective peak areas of the monoesters (i=1), diesters (i=2), triesters (i=3), tetraesters (i=4), pentaesters (i=5) and hexaesters (i=6).
   Aᵢ=sum total of the respective peak areas of the monoesters (i=1), diesters (i=2), triesters (i=3), tetraesters (i=4), pentaesters (i=5) and hexaesters (i=6) in the GC chromatogram [%].

$${n}_{i} = \frac{{a}_{i}}{{M}_{i}}$$

with nᵢ=molar proportion [mol/g] of the respective monoesters (i=1), diesters (i=2), triesters (i=3), tetraesters (i=4), pentaesters (i=5) and hexaesters (i=6).
   Mᵢ=molar mass [g/mol] of the respective monoesters (i=1), diesters (i=2), triesters (i=3), tetraesters (i=4), pentaesters

$$V = {\sum }_{i} \frac{{n}_{i}}{{\sum }_{i} {n}_{i}} \cdot i$$

with V=level of esterification

The inventive n-nonanoic esters of xylitol, sorbitol or erythritol can be prepared by any processes known to the person skilled in the art. If the inventive n-nonanoic esters of xylitol or sorbitol are prepared in the presence of chemical catalysts at relatively high temperatures, at least partial dehydration of xylitol and/or sorbitol can occur.

Three degradation products of xylitol that frequently occur under such conditions are the anhydropentitols 1,4-anhydroxylitol, 1,4-anhydroarabinitol and 1,4-anhydroribitol (J. Carbohydr. Chem. 2004, 23, 4, 169-177 and Adv. Carbohydr. Chem. Biochem., 1983, 41, 27-66).

Four degradation products of sorbitol that frequently occur under such conditions are the anhydrohexitols 1,4-anhydrosorbitol, 2,5-anhydrosorbitol, 1,5-anhydrosorbitol (Advances in Carbohydrate Chemistry and Biochemistry, 1983, 41, 27-66) and isosorbide (1,4:3,6-dianhydrosorbitol; Chem Sus Chem. 5 (1): 167-176).

During the esterification reaction to give the inventive n-nonanoic esters of xylitol or sorbitol, the aforementioned degradation products of xylitol and sorbitol typically likewise afford mono-, di- and triesters of the degradation products, each in the form of mixtures of various regioisomers.

Mixed compositions preferred in accordance with the invention and comprising the n-nonanoic ester according to the invention preferably include such esters of the degradation products of xylitol and sorbitol only in minor amounts.

Thus, all n-nonanoic esters of xylitol, sorbitol, 1,4-anhydrosorbitol, 2,5-anhydrosorbitol, 1,5-anhydrosorbitol, 1,4-anhydroxylitol, 1,4-anhydroarabinitol and 1,4-anhydroribitol, that are present in a mixed composition preferred in accordance with the invention contain a total of less than 20% by weight, preferably less than 15% by weight, particularly preferably less than 10% by weight, especially preferably less than 5% by weight, of residues of 1,4-anhydrosorbitol, 2,5-anhydrosorbitol, 1,5-anhydrosorbitol, 1,4-anhydroxylitol, 1,4-anhydroarabinitol and 1,4-anhydroribitol, where the percentages by weight are based on all residues of xylitol, sorbitol, 1,4-anhydrosorbitol, 2,5-anhydrosorbitol, 1,5-anhydrosorbitol, 1,4-anhydroxylitol, 1,4-anhydroarabinitol and 1,4-anhydroribitol that are present in the aforementioned n-nonanoic esters.

It is alternatively preferred, that high amounts of n-nonanoic esters of the degradation products of xylitol and sorbitol are comprised in the mixed compositions in accordance with the invention. These alternatively preferred mixed compositions in accordance with the invention have outstanding properties in dish washing applications, e.g. they help to reduce deposition of unwanted substances on dishes, especially on metal cutlery.

Thus, all n-nonanoic esters of xylitol, sorbitol, 1,4-anhydrosorbitol, 2,5-anhydrosorbitol, 1,5-anhydrosorbitol, 1,4-anhydroxylitol, 1,4-anhydroarabinitol and 1,4-anhydroribitol, that are present in the alternatively preferred mixed composition in accordance with the invention contain a total of 50% by weight to 95% by weight, preferably of 60% by weight to 90% by weight, particularly preferably of 70% by weight to 85% by weight, of residues of 1,4-anhydrosorbitol, 2,5-anhydrosorbitol, 1,5-anhydrosorbitol, 1,4-anhydroxylitol, 1,4-anhydroarabinitol and 1,4-anhydroribitol, where the percentages by weight are based on all residues of xylitol, sorbitol, 1,4-anhydrosorbitol, 2,5-anhydrosorbitol, 1,5-anhydrosorbitol, 1,4-anhydroxylitol, 1,4-anhydroarabinitol and 1,4-anhydroribitol that are present in the aforementioned n-nonanoic esters.

The content of xylitol, of degradation products of xylitol (1,4-anhydroxylitol, 1,4-anhydroarabinitol and 1,4-anhydroribitol), of sorbitol and of degradation products of sorbitol (1,4-anhydrosorbitol, 2,5-anhydrosorbitol, 1,5-anhydrosorbitol and isosorbide) is determined by means of high-performance liquid chromatography (HPLC). This method includes the alkaline hydrolysis of the n-nonanoic ester to be analysed, removal of the carboxylic acids and analysis of the sugar and sugar alcohol fractions.

For this purpose, an initial charge of 150 mg of the n-nonanoic ester to be analysed in 2.00 ml of an aqueous 1 M KOH solution is hydrolysed while stirring at 95° C. for 30 min. Subsequently, the reaction solution is cooled to room temperature and adjusted to pH 2-3 with a 2 M aqueous HCl solution. The carboxylic acids that precipitate out as a result are then extracted with diethyl ether (3×3.00 ml), with removal of the organic supernatant by pipette after each extraction. After the extraction, the aqueous solution is heated to 50° C. while stirring for 20 min, which removes the rest of the ether (boiling point of diethyl ether: 34.6° C.).

The solution obtained above is made up to 10.0 ml with bidistilled H₂O, then diluted 1:10, and an aliquot of the solution is analysed by means of HPLC. The analysis is conducted under the following conditions:
Column: Aminex HPX-87C column 300×7.8 mm
Eluent: H₂O
Injection volume: 10.0 μl
Flow rate: 0.60 ml/min
Column temperature: 50° C.
Detector: G1362A/1260 RID (from Agilent), 35° C.
Run time: 30.0 min

Xylitol and its degradation products and sorbitol and its degradation products are separated by means of ion exchange processes.

For evaluation, the summated peak areas of xylitol and sorbitol are expressed in relation to the sum total of the peak areas of 1,4-anhydroxylitol, 1,4-anhydroarabinitol and 1,4-anhydroribitol, 1,4-anhydrosorbitol, 2,5-anhydrosorbitol, 1,5-anhydrosorbitol and isosorbide.

Reference substances for the degradation products of xylitol and of sorbitol are commercially available or can alternatively be obtained by heating xylitol and/or sorbitol in form in the presence of acidic (>140° C.) or basic (>180° C.) catalysts.

The present invention thus also further provides a formulation, especially a cosmetic formulation or household care formulation, comprising the inventive n-nonanoic esters of xylitol, sorbitol or erythritol and/or the mixed compositions according to the invention.

The present invention further provides a process for enzymatic preparation of an inventive n-nonanoic ester of xylitol, sorbitol or erythritol according to at least one of claims 1 to 6, comprising the process steps of
A) providing xylitol, sorbitol or erythritol and at least one n-nonanoyl group donor, especially selected from n-nonanoic esters and n-nonanoic acid, more preferably n-nonanoic acid,
B) reacting xylitol, sorbitol or erythritol with the at least one n-nonanoyl group donor in the presence of a lipase at a temperature of 75° C. to 110° C., preferably of 77° C. to 100° C., even more preferably 80° C. to 95° C., to give an n-nonanoic ester of xylitol, sorbitol or erythritol, and optionally
C) purifying the n-nonanoic ester of xylitol, sorbitol or erythritol.

n-Nonanoic esters used with preference in accordance with the invention as acyl group donor are selected from esters based on alkanols and polyols having up to 6 carbon atoms, especially preferably having up to 3 carbon atoms, very preferably glycerol esters.

n-Nonanoic acid which is used with preference in accordance with the invention as acyl group donor may especially be used in the form of technical grade n-nonanoic acid; such technical grade n-nonanoic acids are understood to mean not substances that are of ultra-high purity but those that include a proportion of impurities, for instance in the form of further fatty acids. Particular preference is given to using technical grade n-nonanoic acid having a purity of >85% by weight, preferably >90% by weight, especially preferably >95% by weight, in particular >98% by weight, based on all fatty acids present, which has preferably been obtained proceeding from ω-9-fatty acids, preferably oleic acid and/or erucic acid, especially obtained from non-tropical oils, for example rapeseed oil, sunflower oil and/or safflower oil, in a hydrogen peroxide-based process. Thus, in the process according to the invention, the providing of the n-nonanoic acid in process step A) preferably comprises the additional step of: providing ω-9-fatty acids, preferably oleic acid and/or erucic acid, and reacting these with hydrogen peroxide in the presence of a catalyst, especially tungsten-based catalysts such as tungstic acid and salts thereof, pertungstic acid and salts thereof, tungstophosphoric acid and salts thereof, niobium oxides, cobalt salts such as cobalt acetate and cobalt naphthenate, to give n-nonanoic acid. The ω-9-fatty acids provided, preferably oleic acid and/or erucic acid, have preferably been obtained from non-tropical oils, but rather, for example, from rapeseed oil, sunflower oil and/or safflower oil.

A process preferred in accordance with the invention is characterized in that the xylitol, sorbitol or erythritol and the at least one n-nonanoyl group donor account for at least 80% by weight, preferably at least 90% by weight, especially preferably at least 95% by weight, based on the overall reaction mixture at the start of process step B).

If the reaction mixture includes two or more selected from xylitol, sorbitol and erythritol, these are added together.

Since, in this context, the overall reaction mixture consists largely of the reactants, i.e. xylitol, sorbitol and/or erythritol and n-nonanoyl group donor, only very little solvent—if any—can be present in the overall reaction mixture. It is clear on the basis of the above that the n-nonanoyl group donor is not covered by the term “solvent” in the process according to the invention. Possible solvents would be, for example, ketones, for example methyl isobutyl ketone or cyclohexanone, sterically hindered secondary alcohols such as 2-butyl-1-octanol, methylcyclohexanols, 1-methoxy-2-propanol, butane-2,3-diol, 2-octanol, diacetone alcohol, 2-methyl-2-butanol, and ethers such as 1,4-dioxane, tetrahydrofuran and Varonic® APM.

Based on the overall reaction mixture, solvents are present in a maximum total amount of less than 20% by weight, preferably less than 10% by weight, especially less than 5% by weight. The expression “present in a maximum amount of less than X % by weight” can be equated with “a content is less than X % by weight”.

Particular preference is given to conducting the process according to the invention in a solvent-free manner.

A process which is preferred in accordance with the invention is characterized in that the molar ratio of all hydroxyl groups provided by the xylitol, sorbitol or erythritol provided to n-nonanoyl groups present in all n-nonanoyl group donors provided is within a range from 1.00:0.05 to 1.00:0.90, preferably from 1.00:0.07 to 1.00:0.75, especially preferably from 1.00:0.10 to 1.00:0.50, or alternatively especially preferably from 1.00:0.15 to 1.00:0.35.

If the reaction mixture includes two or more selected from xylitol, sorbitol and erythritol and possibly also further sugars or sugar alcohols (see below), hydroxyl groups provided by these are added up.

A process preferred in accordance with the invention is characterized in that process step A) comprises blending the xylitol, sorbitol or erythritol with the at least one n-nonanoyl group donor for at least ten minutes, preferably 30 minutes, even more preferably 60 minutes, wherein the blending is preferably conducted within a temperature range from 80° C. to 120° C., preferably from 90° C. to 120° C., even more preferably from 95° C. to 120° C., even more preferably from 100° C. to 120° C.

Lipases used with preference in accordance with the invention in process step B) are present immobilized on a solid support.

Lipases used with preference in accordance with the invention in process step B) are lipases selected from the group comprising the lipase from Thermomyces lanuginosus (accession number O59952), lipases A and B (accession number P41365) from Candida antarctica and the lipase from Mucor miehei (accession number P19515), the lipase from Humicola sp. (accession number O59952), the lipase from Rhizomucor javanicus (accession number S32492), the lipase from Rhizopus oryzae (accession number P61872), the lipases from Candida rugosa (accession number P20261, P32946, P32947, P3294 and P32949), the lipase from Rhizopus niveus (accession number P61871), the lipase from Penicillium camemberti (accession number P25234), the lipases from Aspergillus niger (ABG73613, ABG73614 and ABG37906) and the lipase from Penicillium cyclopium (accession number P61869), particular preference being given to lipases A and B (accession number P41365) from Candida antarctica, and their respective at least 60%, with preference at least 80%, preferably at least 90% and especially preferably at least 95%, 98% or 99%, homologues at the amino acid level.

The accession numbers listed in the context of the present invention correspond to the protein bank database entries of the NCBI with a date of 1 Jan. 2017; generally, in the present context, the version number of the entry is identified by “.digit”, for example “0.1”.

The enzymes that are homologous at the amino acid level, by comparison with the reference sequence, preferably have at least 50%, especially at least 90%, enzyme activity in propyl laurate units as defined in the context of the present invention.

In order to determine the enzymatic activity in PLU (propyl laurate units), 1-propanol and lauric acid are mixed homogeneously in an equimolar ratio at 60° C. The reaction is started with addition of enzyme and the reaction time is stopped. Samples are taken from the reaction mixture at intervals and the content of converted lauric acid is determined by means of titration with potassium hydroxide solution. The enzyme activity in PLU results from the rate at which 1 g of the enzyme in question synthesizes 1 μmol of propyl laurate per minute at 60° C.; cf. in this respect also US20070087418, in particular [0185].

Commercial examples, and lipases that are likewise used with preference in processes according to the invention, are the commercial products Lipozyme TL IM, Novozym 435, Lipozyme IM 20, Lipase SP382, Lipase SP525, Lipase SP523, (all commercial products from Novozymes A/S, Bagsvaerd, Denmark), Chirazyme L2, Chirazyme L5, Chirazyme L8, Chirazyme L9 (all commercial products from Roche Molecular Biochemicals, Mannheim, Germany), CALB Immo Plus™ from Purolite, and Lipase M “Amano”, Lipase F-AP 15 “Amano”, Lipase AY “Amano”, Lipase N “Amano”, Lipase R “Amano”, Lipase A “Amano”, Lipase D “Amano”, Lipase G “Amano” (all commercial products from Amano, Japan), Evoxx Lipase 4.3.040 191G immobilized, Evoxx Addzyme CALB 165G immobilized, Evoxx Addzyme TL 165G immobilized, Evoxx Addzyme RD 165G immobilized, Evoxx Addzyme CALB 10P, Evoxx Addzyme CALB 5L, Evoxx Addzyme TL 100P, Evoxx Addzyme TL 100L, Evoxx Addzyme RD 50P, Evoxx Addzyme RD 10L (all commercial products from Evoxx, Germany), Fermenta Biocatalyst CAL B 1L-10L, Fermenta Biocatalyst CAL B 1L-10L, Fermenta Biocatalyst CAL B TA 10000 immobilized, Fermenta Biocatalyst CAL B 1000-5000 immobilized (all commercial products from Fermenta Biotech, India), Purolite CALB Immo 8285 immobilized, Purolite CALB Immo 8806 immobilized, Purolite CALB Immo Kit immobilized, Purolite CALB Immo Plus immobilized (all commercial products from Purolite, USA), Vland L Lipase Kingpase, Vland Kingzyme IM-100, Vland L Lipase Coated Lipase (all commercial products from Vland, China), Clea B1, Eucodis CALB, Eucodis EL001, Eucodis EL012, Eucodis EL013, Eucodis EL016, Eucodis EL056, Eucodis EL070 (all commercial products from Eucodis, Austria).

“Homology at the amino acid level” in the context of the present invention is understood to mean “amino acid identity”, which can be determined with the aid of known methods. In general, use is made of special computer programs with algorithms taking into account specific requirements. Preferred methods for determining the identity initially generate the greatest alignment between the sequences to be compared. Computer programs for determining the identity include, but are not limited to, the GCG program package including
GAP (Deveroy, J. et al., Nucleic Acid Research 12 (1984), page 387, Genetics Computer Group University of Wisconsin, Medicine (WI), and
BLASTP, BLASTN and FASTA (Altschul, S. et al., Journal of Molecular Biology 215 (1990), pages 403-410. The BLAST program can be obtained from the National Center For Biotechnology Information (NCBI) and from other sources (BLAST Handbook, Altschul S. et al., NCBI NLM NIH Bethesda ND 22894; Altschul S. et al., above).

The person skilled in the art is aware that various computer programs are available for the calculation of similarity or identity between two nucleotide or amino acid sequences. For instance, the percentage identity between two amino acid sequences can be determined, for example, by the algorithm developed by Needleman and Wunsch (J. Mol. Biol. (48): 444-453 (1970)), which has been integrated into the GAP program in the GCG software package (available at http://www.gcg.com), using either a Blossom 62 matrix or a PAM250 matrix, a gap weight of 16, 14, 12, 10, 8, 6 or 4 and a length weight of 1, 2, 3, 4, 5 or 6. The person skilled in the art will recognize that the use of different parameters will lead to slightly different results, but that the percentage identity between two amino acid sequences overall will not be significantly different. The Blossom 62 matrix is typically used applying the default settings (gap weight: 12, length weight: 1).

In the context of the present invention, an identity of 60% according to the above algorithm means 60% homology. The same applies to higher identities.

In process step B), preference is given in accordance with the invention to using 25 PLU to 2000 PLU, preferably from 200 PLU to 1500 PLU, especially preferably from 500 PLU to 1250 PLU, of lipase per gram of xylitol, sorbitol or erythritol to be converted.

If the reaction mixture includes two or more selected from xylitol, sorbitol and erythritol and possibly also further sugars or sugar alcohols (see below), the masses thereof are added up.

Preferably in accordance with the invention, process step B) is conducted at a pressure of less than 1 bar, preferably less than 0.5 bar and especially preferably less than 0.1 bar.

Alternatively preferably in accordance with the invention, process step B) is conducted in a bubble column reactor, with at least one inert gas being passed through the reaction mixture; this gas is preferably selected from the group comprising, preferably consisting of, nitrogen and argon. In this context, it is preferable in accordance with the invention for the gas stream to be 1 to 60 kg/h, preferably 5 to 25 kg/h, yet more preferably 10 to 14 kg/h.

Preferably in accordance with the invention, process step B) is characterized in that process step B) is ended no later than 180 hours, preferably 120 hours, especially preferably 100 hours, after the lipase has been added.

A process which is preferred in accordance with the invention is characterized in that by-products formed in process step B), for example water in the case that the n-nonanoyl group donor used is an n-nonanoic acid, the corresponding alcohol in the case that the n-nonanoyl group donor used is an n-nonanoic ester, are removed.

This is possible by distillation for example.

Process step C) of the process according to the invention comprises the purification of the n-nonanoic ester of xylitol, sorbitol or erythritol.

Employable methodologies for this purpose are any that permit the obtaining of the n-nonanoic ester of xylitol, sorbitol or erythritol in higher concentration.

Preferably in accordance with the invention, the process according to the invention comprises, in process step C), removing the lipase used in the process according to the invention.

In the case that the lipase is immobilized on a carrier, it is preferable in accordance with the invention that the lipase is removed by filtration through a filter, especially a bag filter, having a fineness of 0.1μ to 1250μ, preferably of 0.5μ to 200μ, especially preferably 50μ to 100μ.

Preferably in accordance with the invention, the process of the present invention is characterized in that, in process step A), as well as the xylitol, sorbitol or erythritol, at least one other sugar or sugar alcohol is provided, selected from the group of
agarose, allitol, allulose, altritol, amylopectin, amylose, arabinitol, arabinose, cellobiose, cellulose, chitin, cyclodextrins, deoxyribose, dextrans, erythritol, fructans, fructose, fucose, galactitol, galactose, glucitol, glucose, glycogen, hyaluronic acid, iditol, inulin, isomalt, isomaltulose, isomelizitose, lactitol, lactose, lactulose, maltitol, maltohexose, maltopentose, maltose, maltotetrose, maltotriose, maltulose, mannitol, mannose, melizitose, pectins, raffinose, rhamnose, ribitol, ribose, sucrose, sorbitol, sorbose, stachyose, starch, starch hydrolysate, threitol, trehalulose, umbelliferose, xylitol and xylose,
more preferably
allitol, allulose, altritol, arabinitol, arabinose, cellobiose, deoxyribose, erythritol, fructose, fucose, galactitol, galactose, glucitol, glucose, iditol, isomalt, isomaltulose, lactitol, lactose, lactulose, maltitol, maltose, maltulose, mannitol, mannose, rhamnose, ribitol, ribose, sucrose, sorbitol, sorbose, threitol, trehalulose, xylitol and xylose,
very preferably the sugars and sugar alcohols are selected from erythritol, fructose, glucose, isomalt, isomaltulose, lactitol, lactose, maltitol, maltose, maltulose, mannitol, sucrose, sorbitol, sorbose, xylitol and xylose,
especially preferably erythritol, fructose, glucose, sorbitol, xylitol and xylose,
and this also passes through the further process steps.

What is meant by the expression “one other sugar or sugar alcohol” in the context of the present invention is, for example, that when xylitol, for example, is present, what is meant is a sugar or sugar alcohol other than xylitol; the same applies to sorbitol and erythritol.

The present invention further provides the n-nonanoic ester of xylitol, sorbitol or erythritol obtainable by the process according to the invention.

The present invention further provides for the use of the inventive n-nonanoic esters of xylitol, sorbitol or erythritol and/or of the n-nonanoic esters of xylitol, sorbitol or erythritol obtainable by the process according to the invention, and also the mixed compositions according to the invention, as viscosity regulator, active care ingredient, foam booster or solubilizer, antimicrobial, antistat, binder, corrosion inhibitor, dispersant, emulsifier, film former, humectant, opacifier, oral care agent, preservative, skincare agent, hydrophilic emollient, foam stabilizer and/or nonionic surfactant, preferably as viscosity regulator, emulsifier, antimicrobial and/or hydrophilic emollient, especially preferably as viscosity regulator, in particular as thickener, and/or antimicrobial, in particular in cleansing or care formulations.

The examples that follow describe the present invention by way of example, without any intention that the invention, the scope of application of which is apparent from the entirety of the description and the claims, be restricted to the embodiments specified in the examples.

The following figures are an integral part of the examples:

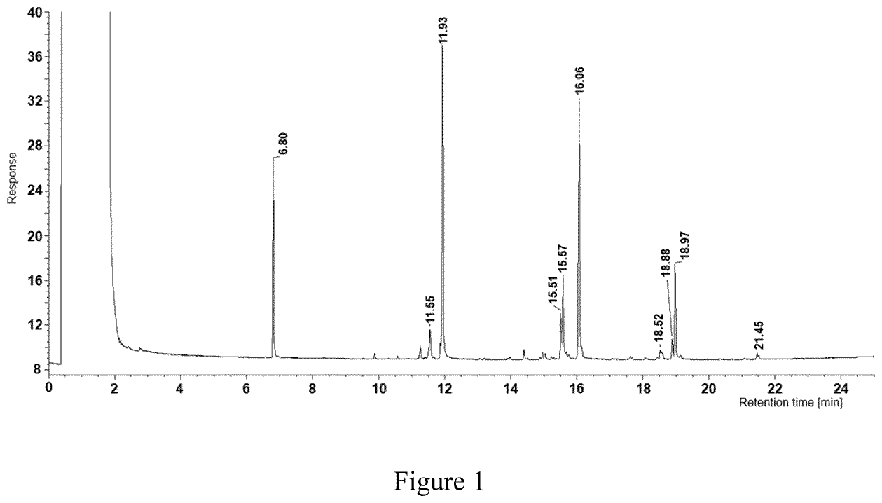

FIG. 1: Gas chromatography of Example 1

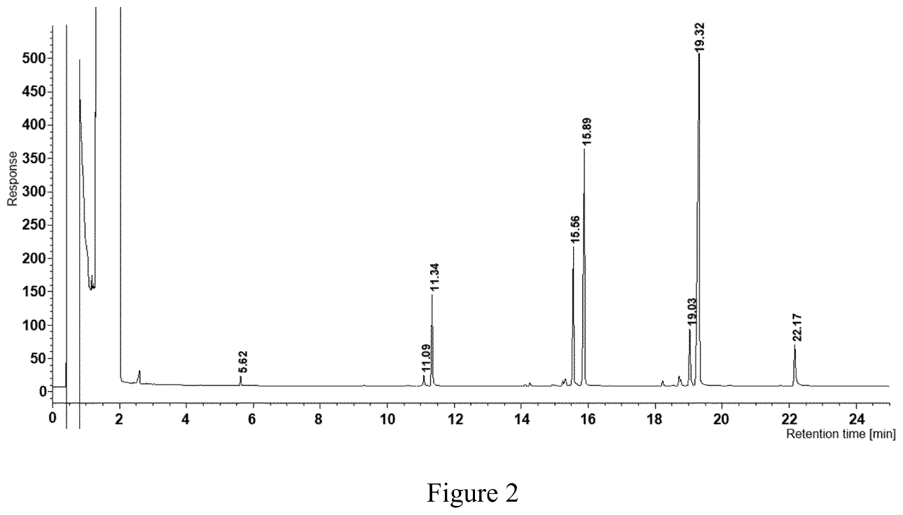

FIG. 2: Gas chromatography of Example 4

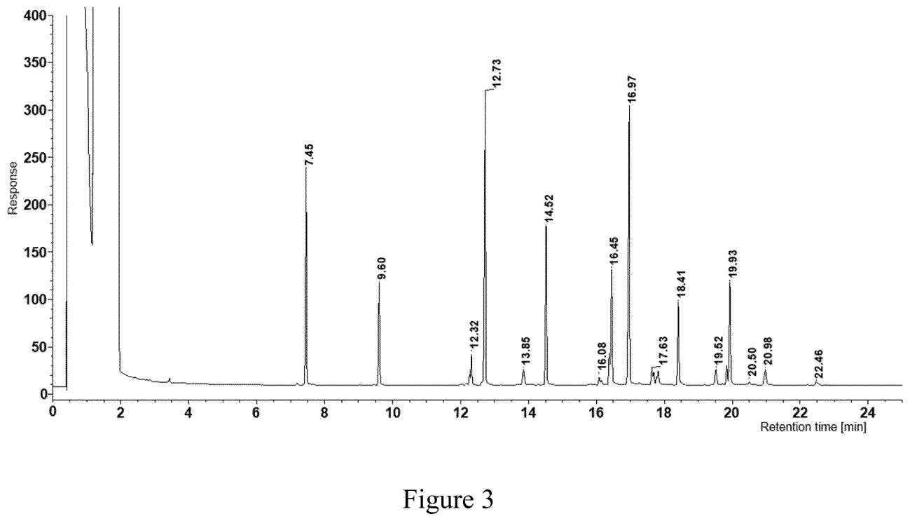

FIG. 3: Gas chromatography of Example 6

### EXAMPLES

### Example 1: Enzymatic Esterification of Xylitol with 1.50 Equiv. of n-Nonanoic Acid (Inventive)

A mixture of xylitol (176.3 g, 1.16 mol, 1.00 equiv.) and n-nonanoic acid (acid number=355 mg KOH/g, 99%, 275.0 g, 1.74 mol, 1.50 equiv.) was heated to 90° C. with stirring and while passing N₂ through, and after 1 h immobilized Candida antarctica lipase B enzyme (13.5 g; Purolite D5619, corresponding to 117234 PLU) was added. The mixture was stirred at 85° C. and 50 mbar for 24 h, during which the water formed was distilled off continuously. Subsequently, the mixture was filtered at 80° C. through a Buchner funnel with black band filter to remove the enzyme. The product obtained was homogeneous in the melt and pale yellowish, and had an acid number of 1.5 mg KOH/g. Analysis by GC-FID showed a mixture of mono-, di- and triesters that each consisted of more than one regioisomer.

This is apparent in FIG. 1: For instance, the signals at 11.55 min and 11.93 min correspond to the regioisomers of the monoester, and the signals at 15.51 min, 15.57 min and 16.06 min to the regioisomers of the diester.

### Example 2: Enzymatic Esterification of a Mixture of 0.90 Equiv. of Xylitol and 0.10 Equiv. of Xylose with 1.50 Eq of n-Nonanoic Acid (Inventive)

A mixture of xylitol (77.0 g, 0.506 mol, 0.90 equiv.), xylose (8.56 g, 0.057 mol, 0.10 equiv.) and n-nonanoic acid (acid number=355 mg KOH/g, 99%, 129.1 g, 0.816 mol, 1.45 equiv.) was heated to 90° C. with stirring and while passing N₂ through, and after 1 h immobilized Candida antarctica lipase B enzyme (6.44 g; Purolite D5619, corresponding to 55925 PLU) was added. The mixture was stirred at 85° C. and 50 mbar for 24 h, during which the water formed was distilled off continuously. Subsequently, the mixture was filtered at 80° C. through a Büchner funnel with black band filter to remove the enzyme. The product obtained was slightly cloudy in the melt and pale yellowish, and had an acid number of 5.6 mg KOH/g. Analysis by GC-FID showed a mixture of mono-, di- and triesters that each consisted of more than one regioisomer.

### Example 3: Enzymatic Esterification of a Mixture of 0.90 Equiv. of Xylitol and 0.10 Equiv. of Xylose with 1.27 Equiv. of n-Nonanoic Acid (Inventive)

A mixture of xylitol (82.9 g, 0.545 mol, 0.90 equiv.), xylose (9.21 g, 0.061 mol, 0.10 equiv.) and n-nonanoic acid (acid number=355 mg KOH/g, 99%, 121.75 g, 0.769 mol, 1.27 equiv.) was heated to 90° C. with stirring and while passing N₂ through, and after 1 h immobilized Candida antarctica lipase B enzyme (6.42 g; Purolite D5619, corresponding to 55751 PLU) was added. The mixture was stirred at 85° C. and 50 mbar for 24 h, during which the water formed was distilled off continuously. Subsequently, the mixture was filtered at 80° C. through a Büchner funnel with black band filter to remove the enzyme. The product obtained was homogeneous in the melt and pale yellowish, and had an acid number of 5.0 mg KOH/g. Analysis by GC-FID showed a mixture of mono-, di- and triesters that each consisted of more than one regioisomer.

### Example 4: Enzymatic Esterification of Erythritol with 1.5 Equiv. of n-Nonanoic Acid (Inventive)

A mixture of erythritol (125.0 g, 1.02 mol, 1.00 equiv.) and n-nonanoic acid (acid number=355 mg KOH/g, 99%, 226.31 g, 1.54 mol, 1.50 equiv.) was heated to 85° C. while stirring and passing N₂ through. After 1 h, immobilized Candida antarctica lipase B enzyme (10.5 g; Purolite D5619, corresponding to 91258 PLU) was added and stirring of the mixture was continued at 85° C. and 15 mbar for 24 h, during which the water formed was distilled off continuously. Subsequently, the mixture was filtered at 80° C. through a Buchner funnel with black band filter to remove the enzyme. The product obtained had an acid number of 5.6 mg KOH/g.

Analysis by GC-FID showed a mixture of mono-, di-, tri- and tetraesters, where the mono-, di- and triesters each consisted of more than one regioisomer.

This is apparent in FIG. 2: The signals at 11.09 min and 11.34 min correspond to the regioisomers of the monoester; the signals at 15.56 min and 15.89 min to the regioisomers of the diester.

### Example 5: Enzymatic Esterification of Sorbitol with 1.55 Equiv. of n-Nonanoic Acid (Inventive)

A mixture of sorbitol (96.5 g, 0.530 mol, 1.00 equiv.) and n-nonanoic acid (acid number=355 mg KOH/g, 99%, 129.9 g, 0.821 mol, 1.55 equiv.) was heated to 100° C. while stirring and passing N₂ through. After 1 h, the mixture was cooled down to 85° C., immobilized Candida antarctica lipase B enzyme (6.79 g; Purolite D5619, corresponding to 58807 PLU) was added and the mixture was stirred further at 85° C. and 15 mbar for 24 h, during which the water formed was continuously distilled off. Subsequently, the mixture was filtered at 80° C. through a Buchner funnel with black band filter to remove the enzyme. The product obtained had an acid number of 3.2 mg KOH/g. Analysis by GC-FID showed a mixture of mono-, di-, tri- and tetraesters that each consisted of more than one regioisomer.

### Example 5a: Enzymatic Esterification of Sorbitol with 2.90 Equiv. of n-Nonanoic Acid (Inventive)

A mixture of sorbitol (96.5 g, 0.530 mol, 1.00 equiv.) and n-nonanoic acid (acid number=355 mg KOH/g, 99%, 243.2 g, 1.54 mol, 2.90 equiv.) was heated to 100° C. while stirring and passing N₂ through. After 1 h, the mixture was cooled down to 85° C., immobilized Candida antarctica lipase B enzyme (10.2 g; Purolite D5619, corresponding to 88236 PLU) was added and the mixture was stirred further at 85° C. and 15 mbar for 24 h, during which the water formed was continuously distilled off. Subsequently, the mixture was filtered at 80° C. through a Büchner funnel with black band filter to remove the enzyme. The product obtained had an acid number of 3.9 mg KOH/g. Analysis by GC-FID showed a mixture of mono-, di-, tri- and tetraesters that each consisted of more than one regioisomer.

### Example 6: Enzymatic Esterification of a Mixture of 0.74 Equiv. of Xylitol and 0.26 Equiv. of Sorbitol with 1.30 Eq of n-Nonanoic Acid (Inventive)

A mixture of xylitol (65.5 g, 0.430 mol, 0.74 equiv.), sorbitol (28.1 g, 0.154 mol, 0.26 equiv.) and n-nonanoic acid (acid number=355 mg KOH/g, 99%, 120.2 g, 0.759 mol, 1.30 equiv.) was heated to 90° C. with stirring and while passing N₂ through, and after 1 h immobilized Candida antarctica lipase B enzyme (6.41 g; Purolite D5619, corresponding to 55500 PLU) was added. The mixture was stirred at 85° C. and 50 mbar for 24 h, during which the water formed was distilled off continuously. Subsequently, the mixture was filtered at 80° C. through a Büchner funnel with black band filter to remove the enzyme. The product obtained had an acid number of 1.5 mg KOH/g. Analysis by GC-FID showed a mixture of mono-, di-, tri- and tetraesters that each consisted of more than one regioisomer.

This is apparent in FIG. 3: The signals at 12.32 min and 12.73 min correspond to the regioisomers of the xylityl monoester; the signals at 13.85 min and 14.52 min correspond to the regioisomers of the sorbityl monoester; the signals at 16.08 min, 16.45 min and 16.97 min correspond to the regioisomers of the xylityl diesters; the signals at 17.63 min and 18.41 min correspond to the regioisomers of the sorbityl diester.

### Example 7: Enzymatic Esterification of Xylitol with 1.50 Equiv. of Caprylic/Capric Acid (Non-Inventive)

A mixture of xylitol (75.7 g, 0.497 mol, 1.00 equiv.) and a mixture of caprylic acid and capric acid (acid number=362 mg KOH/g, mixing ratio of caprylic acid to capric acid 60:40, 115.7 g, 0.746 mol, 1.50 equiv.) was heated to 90° C. with stirring and while passing N₂ through for 1 h and, after cooling to 85° C., immobilized Candida antarctica lipase B enzyme (5.74 g; Purolite D5619, corresponding to 49710 PLU) was added. The mixture was stirred at 85° C. and 50 mbar for 24 h, during which the water formed was distilled off continuously. Subsequently, the mixture was filtered at 80° C. through a Büchner funnel with black band filter to remove the enzyme. The product obtained had an acid number of 1.5 mg KOH/g.

### Example 8: Enzymatic Esterification of a Mixture of 0.74 Equiv. of Xylitol and 0.26 Equiv. of Sorbitol with 1.30 Eq of Caprylic/Capric Acid (Noninventive)

A mixture of xylitol (131.5 g, 0.864 mol, 0.74 equiv.), sorbitol (56.4 g, 0.309 mol, 0.26 equiv.) and a mixture of caprylic acid and capric acid (acid number=362 mg KOH/g, mixing ratio of caprylic acid to capric acid 60:40, 239.6 g, 1.53 mol, 1.30 equiv.) was heated to 90° C. with stirring and while passing N₂ through, and after 30 min immobilized Candida antarctica lipase B enzyme (12.8 g; Purolite D5619, corresponding to 110827 PLU) was added. The mixture was subsequently stirred at 80° C. and 20 mbar for 24 h, during which the water formed was continuously distilled off. Subsequently, the mixture was filtered at 80° C. through a Büchner funnel with black band filter to remove the enzyme. The product obtained had an acid number of 3.0 mg KOH/g.

### Examples 9a to 9f: Chemical Esterification of Xylitol and Sorbitol (Inventive)

Xylitol or sorbitol (or aqueous solutions thereof) were initially charged together with n-nonanoic acid and, after the catalyst had been added, the reaction mixture was heated to reaction temperature while stirring at the pressure specified within 1 h, and the water formed was removed continuously until the acid number specified had been attained. Finally, the mixture was filtered through a filter press.

TABLE 1

| | | | | | | | | | Acid |
|---|---|---|---|---|---|---|---|---|---|
| | | Sugar | Sugar | Pelargonic | Pelargonic | Pelargonic | | | number of |
| | Sugar | alcohol | alcohol | acid | acid | acid | | Reaction | the product |
| | alcohol | m [g] | n [mol] | m/g | n [mol] | Equiv. | Catalyst | conditions | [mg KOH/g] |
| 9a | Xylitol | 290.1 | 1.91 | 467.6 | 2.96 | 1.55 | 3.7 g H₃PO₄ | 230° C., | 7.9 |
| | | | | | | | and 6.6 g | 1 atm | |
| | | | | | | | NaOH | | |
| 9b | Xylitol | 290.0 | 1.91 | 392.1 | 2.48 | 1.30 | 0.8 g para- | 160° C., | 5.7 |
| | | | | | | | toluenesulfonic | 1 atm | |
| | | | | | | | acid | | |
| 9c | Xylitol | 275.0 | 1.81 | 486.2 | 3.07 | 1.70 | 19.4 g K₂CO₃ | 180° C., | 7.1 |
| | | | | | | | | 50 mbar | |
| 9d | Sorbitol | 390.5 | 1.50 | 368.0 | 2.33 | 1.55 | 2.9 g H₃PO₄ | 230° C., | 8.6 |
| | (70% aqueous | | | | | | and 5.0 g | 1 atm | |
| | solution) | | | | | | NaOH | | |
| 9e | Sorbitol | 389.7 | 1.50 | 308.0 | 1.95 | 1.30 | 0.6 g para- | 160° C., | 5.6 |
| | (70% aqueous | | | | | | toluenesulfonic | 1 atm | |
| | solution) | | | | | | acid | | |
| 9f | Sorbitol | 370.2 | 1.42 | 382.6 | 2.42 | 1.70 | 15.3 g K₂CO₃ | 180° C., | 6.9 |
| | (70% aqueous | | | | | | | 50 mbar | |
| | solution) | | | | | | | | |

### Example 9g: Preparation of Xylitol Caprylate (=Xylitol Octanoate) in Analogy to Example 3 from WO94/12651A1 (Non-Inventive)

A mixture of xylitol (0.5 g, 3.3 mmol) and octanoic acid (99%, 3.35 g, 23.2 mmol) was heated to 50° C. under mechanical stirring. Then, sodium octanoate (0.85 g, 5.1 mmol) was added as well as Candida antarctica lipase B enzyme (0.5 mL of an aqueous solution containing 5000 PLU/mL) and the mixture was subsequently stirred at 50° C. for 20 h, Subsequently, the mixture was filtered at 50° C. through a Buchner funnel with black band filter.

### Example 10: Thickening Performance in a Cosmetic Formulation at Relatively Low Concentration

The thickening effect of inventive examples 1 and 4 was evaluated in comparison with non-inventive thickeners. For this purpose, a cosmetic formulation consisting of 4.8% Cocoamphoacetate, 4.8% Cocamidopropyl Betaine, 3.6% Sodium Lauroyl Sarcosinate in water was produced. The pH of this formulation was adjusted to 5.2 with citric acid. 0.6% of the abovementioned example substances was incorporated into each of these formulations at 60° C. by stirring for 30 min, and the viscosities were measured with the aid of a Brookfield viscometer (spindle 62, 30 rpm) at 22° C. The results of the viscosity measurements are shown in Table 2.

TABLE 2

| | Reference | Composition | Viscosity |
|---|---|---|---|
| Inventive | Example 1 | Xylityl nonanoate | 5269 |
| | | | mPa · s |
| Inventive | Example 4 | Erythrityl nonanoate | 4289 |
| | | | mPa · s |
| Inventive | Example 5 | Sorbityl nonanoate | 4401 |
| | | | mPa · s |
| Non- | Example 7 | Xylityl caprylate caprate | 5216 |
| inventive | | | mPa · s |
| Non- | as per | Sorbitan sesquicaprylate | 4245 |
| inventive | DE102009001748A | | mPa · s |
| Non- | as per EP879872 | Sorbityl hexanonanoate | 150 |
| inventive | | | mPa · s |
| Non- | as per Goodby et | 1-O-Nonanoyl-D,L-xylitol | 3951 |
| inventive | al. in Liquid | | mPa · s |
| | Crystals 1997, 22, | | |
| | 367-378 | | |
| Non- | as per example 3 | Xylitol Caprate (=Xylitol | 1589 |
| inventive | of WO94/12651A1 | decanoate) | mPa · s |
| Non- | Example 9g | Xylitol Caprylate (=Xylitol | 874 |
| inventive | | octanoate) | mPa · s |

### Example 11: Thickening Performance in a Cosmetic Formulation at Relatively High Concentration

The thickening effect of inventive examples 3, 4, 5 and 6 was evaluated in comparison with non-inventive thickeners. For this purpose, a cosmetic formulation consisting of 4.8% Cocoamphoacetate, 4.8% Cocamidopropyl Betaine, 3.6% Sodium Lauroyl Sarcosinate in water was produced. The pH of this formulation was adjusted to 5.2 with citric acid. 0.8% of the abovementioned example substances was incorporated into each of these formulations at 60° C. by stirring for 30 min, and the viscosities were measured with the aid of a Brookfield viscometer (spindle 62, 30 rpm) at 22° C. The results of the viscosity measurements are shown in Table 3.

TABLE 3

| | Reference | Composition | Viscosity |
|---|---|---|---|
| Inventive | Example 3 | Xylityl xylose nonanoate | 2902 |
| | | | mPa · s |
| Inventive | Example 4 | Erythrityl nonanoate | 2853 |
| | | | mPas |
| Inventive | Example 5 | Sorbityl nonanoate | 3010 |
| | | | mPas |
| Inventive | Example 6 | Xylityl sorbityl | 3392 |
| | | nonanoate | mPas |
| Non- | Example 8 | Xylityl sorbityl caprylate/ | 2709 |
| inventive | | caprate | mPas |
| Non- | as per | Sorbitan sesquicaprylate | 2763 |
| inventive | DE102009001748A | | mPas |
| Non- | as per EP879872 | Sorbityl hexanonanoate | 705 |
| inventive | | | mPas |
| Non- | as per Goodby et | 1-O-Nonanoyl-D,L-xylitol | 2703 |
| inventive | al. in Liquid | | mPas |
| | Crystals 1997, 22, | | |
| | 367-378 | | |
| Non- | as per example 3 | Xylitol Caprate (=Xylitol | 800 |
| inventive | of WO94/12651A1 | decanoate) | mPas |
| Non- | Example 9g | Xylitol Caprylate | 1141 |
| inventive | | (=Xylitol octanoate) | mPas |

### Example 12: Thickening Performance in a Cosmetic Formulation

The thickening effect of inventive examples 1, 4, 5 and 6 was evaluated in comparison with non-inventive thickeners. For this purpose, a cosmetic formulation consisting of 9% SLES, 3% Cocamidopropyl Betaine and 0.7% NaCl in water was produced. The pH of this formulation was adjusted to 5.2 with citric acid. 1.1% of the abovementioned example substances was incorporated into each of these formulations at 60° C. by stirring for 30 min, and the viscosities were measured with the aid of a Brookfield viscometer (spindle 62, 30 rpm) at 22° C. The results of the viscosity measurements are shown in Table 4.

TABLE 4

| | Reference | Composition | Viscosity |
|---|---|---|---|
| Inventive | Example 1 | Xylityl | 3157 |
| | | nonanoate | mPas |
| Inventive | Example 4 | Erythrityl | 3100 |
| | | nonanoate | mPas |
| Inventive | Example 5 | Sorbityl | 3213 |
| | | nonanoate | mPas |
| Inventive | Example 6 | Xylityl sorbityl | 4843 |
| | | nonanoate | mPas |
| Non-inventive | as per | Xylitan | 3061 |
| | KR101939851B1 | Sesquicaprylate | mPas |
| Non-inventive | as per | Sorbityl | 50 |
| | EP879872 | hexanonanoate | mPas |
| Non-inventive | as per Goodby | 1-O-Nonanoyl- | 2974 |
| | et al. in Liquid | D,L-xylitol | mPas |
| | Crystals 1997, | | |
| | 22, 367-378 | | |

### Example 13: Hand Wash Test

To evaluate the skin feel during the washing a test was performed with a trained sensory panel. The formulations from Example 10 were used in sensory hand wash test. For this purpose, the group of at least 10 trained test persons washed their hands according to a well-defined procedure. Before application the hands have to be cleaned before the test in a standardized way with 2 g of a standard surfactant solution for 10 seconds and the formulation is rinsed off for 10 seconds. After this pre-washing step 2 g of the formulation containing the given composition were applied on the wet palm of a hand. Foam is generated between both hands and the skin feel during washing is judged on a grading scale from 1 (very bad) to 5 (very good). The formulation is rinsed off for 15 seconds. Afterwards two separate judgements for the skin smoothness and the skin softness are given on a grading scale from 1 (very bad) to 5 (very good). This is performed directly after drying and after 3 minutes.

TABLE 5

| | | | | | | skin | skin | |
|---|---|---|---|---|---|---|---|---|
| | | | skin | skin | skin | softness | smoothness | |
| | | | feel | softness | smoothness | after | after | |
| | | | during | after | after | 3 | 3 | Total |
| | Reference | Composition | washing | washing | washing | min | min | Score |
| Inventive | Example 1 | Xylityl nonanoate | 3.0 | 3.2 | 3.0 | 3.2 | 3.2 | 3.1 |
| Inventive | Example 4 | Erythrityl nonanoate | 3.2 | 3.5 | 3.4 | 3.6 | 3.6 | 3.43 |
| Inventive | Example 5 | Sorbityl nonanoate | 2.9 | 3.1 | 3.3 | 3.4 | 3.5 | 3.2 |
| Inventive | Example 5a | Sorbityl nonanoate | 2.7 | 2.8 | 2.5 | 2.3 | 2.5 | 2.56 |
| Non- | Example 7 | Xylityl caprylate caprate | 2.0 | 2.1 | 2.1 | 2.2 | 2.4 | 2.12 |
| inventive | | | | | | | | |
| Non- | as per | Sorbitan sesquicaprylate | 1.8 | 1.8 | 2.0 | 2.0 | 1.8 | 1.86 |
| inventive | DE102009001748A | | | | | | | |
| Non- | as per EP879872 | Sorbityl hexanonanoate | 2.0 | 1.9 | 2.2 | 1.8 | 2.2 | 1.99 |
| inventive | | | | | | | | |
| Non- | as per Goodby et al. in | 1-O-Nonanoyl-d.I-xylitol | 1.4 | 1.6 | 1.7 | 1.5 | 1.5 | 1.51 |
| inventive | Liquid Crystals | | | | | | | |
| | 1997. 22. 367-378 | | | | | | | |
| Non- | as per WO94/12651A1 | Xylitol Caprate (=Xylitol | 1.8 | 2.1 | 2.2 | 2.0 | 2.0 | 1.99 |
| inventive | | decanoate) | | | | | | |
| Non- | Example 9g | Xylitol Caprylate | 2.0 | 1.7 | 1.7 | 2.0 | 2.0 | 1.84 |
| inventive | | (=Xylitol octanoate) | | | | | | |
| Non- | As per example 17 of | Erythrityl nonanoate | 2.0 | 1.8 | 1.8 | 2.0 | 2.0 | 1.92 |
| inventive | WO2020116411A1 | (Hydroxyl value: | | | | | | |
| | | 40 mg KOH/g) | | | | | | |

It can be seen from the measurement results in table 5 that washing hands with the formulations according to the invention using the compositions according to the invention causes the highest score for skin feel after application.

### Formulation Examples

### Recipes 1a, 1b, 1c and 1d: Shower Cream

| | 1a | 1b | 1c | 1d |
|---|---|---|---|---|
| Water | to 100.0% | to 100.0% | to 100.0% | to 100.0% |
| Composition from Example 1 | 1.5% | | | |
| Composition from Example 3 | | 1.5% | | |
| Composition from Example 4 | | | 1.5% | |
| Composition from Example 5 | | | | 1.5% |
| Sodium Laureth Sulfate (Texapon NSO, BASF, 28%) | 25.0% | 25.0% | 25.0% | 25.0% |
| Coco-Glucoside (Plantacare 818 UP, BASF, 51%) | 8.0% | 8.0% | 8.0% | 8.0% |
| Cocamidopropyl Betaine (TEGO ® Betain F 50, Evonik, | 8.0% | 8.0% | 8.0% | 8.0% |
| 38%) | | | | |
| PEG-18 Glyceryl Oleate/Cocoate (ANTIL ® 171, Evonik) | 1.5% | 1.5% | 1.5% | 1.5% |
| Sorbitan Sesquicaprylate (ANTIL ® Soft SC, Evonik) | 0.8% | 0.8% | 0.8% | 0.8% |
| Glyceryl Oleate (TEGIN ® O V, Evonik) | 0.8% | 0.8% | 0.8% | 0.8% |
| Perfume Spicy Herbs (IFF) | 0.2% | 0.2% | 0.2% | 0.2% |
| Polyglyceryl-4 Caprate (TEGOSOFT ® PC 41, Evonik) | 0.6% | 0.6% | 0.6% | 0.6% |
| Helianthus Annuus Seed Oil (AEC Sunflower Oil, A & E | 0.2% | 0.2% | 0.2% | 0.2% |
| Connock, Perfumery & Cosmetics Ltd.) | | | | |
| Linalool (Lipofresh, Lipo Chemicals, Inc,) | 0.1% | 0.1% | 0.1% | 0.1% |
| Coumarin (Rhodiascent extra pure, Solvay Rhodia) | 0.1% | 0.1% | 0.1% | 0.1% |
| Glycerol (Glycerol EP, vegetable, Spiga Nord) | 0.4% | 0.4% | 0.4% | 0.4% |
| Hydroxypropyl Methylcellulose (TEGOCEL ® HPM 50, | 0.2% | 0.2% | 0.2% | 0.2% |
| Evonik) | | | | |
| Glycol Distearate (TEGIN ® G 1100 Pellets, Evonik) | 0.4% | 0.4% | 0.4% | 0.4% |
| Sodium Chloride | 0.5% | 0.5% | 0.5% | 0.5% |
| Hydroxypropyl Guar Hydroxypropyltrimonium Chloride | 0.2% | 0.2% | 0.2% | 0.2% |
| (Jaguar C-162, Solvay Rhodia) | | | | |
| dermofeel TOCO 70 non GMO | 0.1% | 0.1% | 0.1% | 0.1% |
| Disodium EDTA (Dissolvine NA-2-P, AkzoNobel) | 0.1% | 0.1% | 0.1% | 0.1% |
| Preservative | q.s. | q.s. | q.s. | q.s. |
| Citric Acid | to pH 5.2 | to pH 5.2 | to pH 5.2 | to pH 5.2 |

### Recipes 2a, 2b, 2c and 2d: Body Shampoo

| | | 2a | 2b | 2c | 2d |
|---|---|---|---|---|---|
| Phase | Composition from Example 1 | 0.5% | | | |
| A | Composition from Example 3 | | 0.5% | | |
| | Composition from Example 4 | | | 0.5% | |
| | Composition from Example 5 | | | | 0.5% |
| | Lavandula Angustifolia (Lavender) Oil (AEC | 0.2% | 0.2% | 0.2% | 0.2% |
| | Lavender Oil, A&E Connock Ltd.) | | | | |
| | Perfume | 0.1% | 0.1% | 0.1% | 0.1% |
| Phase | Sodium Cocoamphoacetate (REWOTERIC ® AM | 10.0% | 10.0% | 10.0% | 10.0% |
| B | C, Evonik, 32%) | | | | |
| Phase | Water | to 100.0% | to 100.0% | to 100.0% | to 100.0% |
| C | Xanthan Gum (Keltrol CG-SFT, CP Kelco) | 1.2% | 1.2% | 1.2% | 1.2% |
| Phase | Sodium Lauroyl Methyl Isethionate (Iselux, | 4.5% | 4.5% | 4.5% | 4.5% |
| D | Innospec Active Chemicals) | | | | |
| | Capryl/Capramidopropyl Betaine (TEGO ® | 4.5% | 4.5% | 4.5% | 4.5% |
| | Betaine 810, Evonik, 38%) | | | | |
| | Citric Acid | 1.2% | 1.2% | 1.2% | 1.2% |
| Phase | Water | 10.0% | 10.0% | 10.0% | 10.0% |
| E | Polyquaternium-7 (Merquat 550, Nalco) | 0.4% | 0.4% | 0.4% | 0.4% |
| | Preservative | q.s. | q.s. | q.s. | Body |
| | | | | | cream |

### Recipes 3a, 3b, 3c and 3d: Shampoo

| | | 3a | 3b | 3c | 3d |
|---|---|---|---|---|---|
| Phase | Composition from Example 1 | 3.5% | | | |
| A | Composition from Example 3 | | 3.5% | | |
| | Composition from Example 4 | | | 3.5% | |
| | Composition from Example 5 | | | | 3.5% |
| | Isopropyl Myristate (TEGOSOFT ® M, | 0.2% | 0.2% | 0.2% | 0.2% |
| | Evonik) | | | | |
| | Perfume | 0.1% | 0.1% | 0.1% | 0.1% |
| Phase | Water | to 100.0% | to 100.0% | to 100.0% | to 100.0% |
| B | | | | | |
| Phase | Sodium Lauryl Sulfate (Texapon LS 35, | 28.0% | 28.0% | 28.0% | 28.0% |
| C | BASF, 30%) | | | | |
| Phase | Cocamidopropyl Betaine (TEGO ® | 9.0% | 9.0% | 9.0% | 9.0% |
| D | Betain F 50, Evonik, 38%) | | | | |
| Phase | Cocamide MEA (REWOMID ® C 212, | 2.0% | 2.0% | 2.0% | 2.0% |
| E | Evonik) | | | | |
| | Xanthan Gum (Keltrol CG-SFT, CP | 0.3% | 0.3% | 0.3% | 0.3% |
| | Kelco) | | | | |
| | Water | 10.0% | 10.0% | 10.0% | 10.0% |
| Phase | Water | 10.0% | 10.0% | 10.0% | 10.0% |
| F | Polyquaternium-10 (Polymer JR 400, | 0.2% | 0.2% | 0.2% | 0.2% |
| | Amerchol) | | | | |
| Phase | Citric Acid | to pH 5.0 | to pH 5.0 | to pH 5.0 | to pH 5.0 |
| G | | | | | |
| Phase | Preservative | q.s. | q.s. | q.s. | Body |
| H | | | | | cream |

### Recipes 4a, 4b, 4c and 4d: Shampoo

| Recipes | 4a | 4b | 4c | 4d |
|---|---|---|---|---|
| Water | to 100.0% | to 100.0% | to 100.0% | to 100.0% |
| Composition from Example 1 | 2.5% | 2.5% | | |
| Composition from Example 3 | 1.5% | 1.5% | | |
| Composition from Example 4 | | | 2.5% | 2.5% |
| Composition from Example 5 | | | 1.5% | 1.5% |
| Cocamidopropyl Betaine (TEGO ® Betain F 50, | 22.0% | 22.0% | 22.0% | 22.0% |
| Evonik, 38%) | | | | |
| Lauryl Glucoside (Plantacare 1200 UP, BASF, 50%) | 6.0% | 6.0% | 6.0% | 6.0% |
| Sodium Cocoyl Glutamate (Plantapon ACG HC, | 1.5% | 1.5% | 1.5% | 1.5% |
| BASF) | | | | |
| Sodium Cocoyl Glycinate (Hostapon SG, Clariant) | 0.8% | 0.8% | 0.8% | 0.8% |
| Zinc Pyrithione (Microcare ZP, Thor) | 0.1% | 0.1% | 0.1% | 0.1% |
| PEG-120 Methyl Glucose Dioleate (ANTIL ® 120 | 0.4% | 0.4% | 0.4% | 0.4% |
| Plus, Evonik) | | | | |
| Sodium Chloride | 0.5% | 0.5% | 0.5% | 0.5% |
| Isostearamide MIPA; Glyceryl Laurate (ANTIL ® SPA | 0.5% | 0.5% | 0.5% | 0.5% |
| 80, Evonik) | | | | |
| Xanthan Gum (Keltrol CG-SFT, CP Kelco) | 0.3% | 0.3% | 0.3% | 0.3% |
| Hydroxypropyl Guar Hydroxypropyltrimonium | 0.3% | 0.3% | 0.3% | 0.3% |
| Chloride (Jaguar C-162, Solvay Rhodia) | | | | |
| Quaternium-80 (ABIL ® Quat 3272, Evonik) | 0.4% | 0.4% | 0.4% | 0.4% |
| Palmitamidopropyltrimonium Chloride (VARISOFT ® | 0.4% | 0.4% | 0.4% | 0.4% |
| PATC, Evonik) | | | | |
| Argania Spinosa Oil (Argan Oil, DSM Nutritional | 0.1% | 0.1% | 0.1% | 0.1% |
| Products Ltd.) | | | | |
| Glycerol (Glycerol EP, vegetable, Spiga Nord) | 0.6% | 0.6% | 0.6% | 0.6% |
| Tetrasodium EDTA (Versene 100, The Dow | 0.1% | 0.1% | 0.1% | 0.1% |
| Chemical Company) | | | | |
| Caffeine (Merck KGaA/EMD Chemicals, Inc.) | 0.1% | 0.1% | 0.1% | 0.1% |
| Hydrolyzed Wheat Protein (Gluadin WLM, BASF) | 0.1% | 0.1% | 0.1% | 0.1% |
| Limonene (Dipentene No, 122, Hercules Inc.) | 0.1% | 0.1% | 0.1% | 0.1% |
| Citric Acid | to pH 5.5 | to pH 5.5 | to pH 5.5 | to pH 5.5 |
| Sodium Phytate; Aqua; Alcohol (dermofeel ® PA-3; | | 0.1% | 0.1% | 0.1% |
| Evonik Dr, Straetmans GmbH (PA-3) | | | | |
| Perfume | 0.2% | | | |
| Preservative | q.s. | | | |
| Aqua; Sodium Levulinate; Sodium Benzoate | | 1.2% | 1.2% | 1.2% |
| (Verstatil ® BL non GMO; Evonik Dr. Straetmans | | | | |
| GmbH) | | | | |

### Recipes 5a, 5b, 5c and 5d: Liquid Soap

| | 5a | 5b | 5c | 5d |
|---|---|---|---|---|
| Water | to 100% | to 100.0% | to 100.0% | to 100.0% |
| Glycerol (Glycerol EP, vegetable, Spiga Nord) | 4.0% | 4.0% | 4.0% | 4.0% |
| Alcohol | 4.0% | 4.0% | 4.0% | 4.0% |
| Sodium Coco-Sulfate (Texapon HC G, BASF) | 3.0% | 3.0% | 3.0% | 3.0% |
| Lauryl Glucoside (Plantacare 1200 UP, BASF, 50%) | 6.0% | 6.0% | 6.0% | 6.0% |
| Composition from Example 1 | 0.1% | | | |
| Composition from Example 3 | | 0.1% | | |
| Composition from Example 4 | | | 0.1% | |
| Composition from Example 5 | | | | 0.1% |
| Xanthan Gum (Keltrol CG-SFT, CP Kelco) | 1.5% | 1.5% | 1.5% | 1.5% |
| Mangifera Indica (Mango) Fruit Extract (Mango | 0.5% | 0.5% | 0.5% | 0.5% |
| Extract, Draco Natural Products) | | | | |
| Limonene (Dipentene No, 122, Hercules Inc.) | 0.1% | 0.1% | 0.1% | 0.1% |
| Linalool (Lipofresh, Lipo Chemicals, Inc.) | 0.1% | 0.1% | 0.1% | 0.1% |
| Citric Acid | to pH 4.9 | to pH 4.9 | to pH 4.9 | to pH 4.9 |
| Preservative | q.s. | q.s. | q.s. | q.s. |
| Dyes | Body | q.s. | q.s. | q.s. |
| | cream | | | |

### Recipes 6a, 6b, 6c and 6d: Cream Soap

| | 6a | 6b | 6c | 6d |
|---|---|---|---|---|
| Water | to 100% | to 100% | to 100% | to 100% |
| Propylene Glycol (Euxyl K 320, Schulke & Mayr | 2.0% | 2.0% | 2.0% | 2.0% |
| GmbH) | | | | |
| Coco-Glucoside (Plantacare 818 UP, BASF, 51%) | 10.0% | 10.0% | 10.0% | 10.0% |
| Glycerol (Glycerol EP, vegetable, Spiga Nord) | 5.0% | 5.0% | 5.0% | 5.0% |
| Composition from Example 1 | 2.5% | | | |
| Composition from Example 3 | | 2.5% | | |
| Composition from Example 4 | | | 2.5% | |
| Composition from Example 5 | | | | 2.5% |
| Disodium Cocoyl Glutamate (Planatpon ACG LC, | 2.5% | 2.5% | 2.5% | 2.5% |
| BASF) | | | | |
| Xanthan Gum (Keltrol CG-SFT, CP Kelco) | 1.2% | 1.2% | 1.2% | 1.2% |
| Stearic Acid (Pristerene 4922, Croda Europe, Ltd.) | 1.2% | 1.2% | 1.2% | 1.2% |
| Citric Acid | to pH 5.5 | to pH 5.5 | to pH 5.5 | to pH 5.5 |
| Olea Europaea Fruit Oil (Cropure Olive, Croda | 0.2% | 0.2% | 0.2% | 0.2% |
| Europe, Ltd.) | | | | |
| Glyceryl Oleate (TEGIN ® O V, Evonik) | 1.0% | 1.0% | 1.0% | 1.0% |
| Sodium Cocoyl Glutamate (Plantapon ACG HC, | 0.8% | 0.8% | 0.8% | 0.8% |
| BASF) | | | | |
| Tetrasodium EDTA (Versene 100, The Dow | 0.2% | 0.2% | 0.2% | 0.2% |
| Chemical Company) | | | | |
| Perfume | 0.1% | 0.1% | 0.1% | 0.1% |
| Preservative | q.s. | q.s. | q.s. | q.s. |
| Dyes | q.s. | q.s. | q.s. | Body |
| | | | | cream |

### Recipes 7a, 7b, 7c and 7d: Oil Bath

| | 7a | 7b | 7c | 7d |
|---|---|---|---|---|
| Water | to 100.0% | to 100.0% | to 100.0% | to 100.0% |
| Glycine Soja Oil (Cropure Soybean, Croda Europe, | 20.0% | 20.0% | 20.0% | 20.0% |
| Ltd.) | | | | |
| Composition from Example 1 | 12.0% | | | |
| Composition from Example 3 | | 12.0% | | |
| Composition from Example 4 | | | 12.0% | |
| Composition from Example 5 | | | | 12.0% |
| Polyglyceryl-3 Palmitate (Dermofeel ® PP, Evonik | 4.5% | 4.5% | 4.5% | 4.5% |
| Dr. Straetmans) | | | | |
| Glyceryl Caprylate (Dermosoft ® GMCY, Evonik Dr. | 3.0% | 3.0% | 3.0% | 3.0% |
| Straetmans) | | | | |
| Simmondsia Chinensis Seed Oil (AEC Jojoba Oil | 1.2% | 1.2% | 1.2% | 1.2% |
| Refined, A & E Connock, Perfumery & Cosmetics | | | | |
| Ltd.) | | | | |
| Prunus Amygdalus Dulcis (Sweet Almond) Oil | 1.0% | 1.0% | 1.0% | 1.0% |
| (Cropure Almond, Croda Europe, Ltd.) | | | | |
| Triticum Vulgare Germ Oil (Cropure Wheatgerm, | 0.5% | 0.5% | 0.5% | 0.5% |
| Croda Europe, Ltd.) | | | | |
| Tocopherol (Euxyl K 700, Schülke & Mayr GmbH) | 0.2% | 0.2% | 0.2% | 0.2% |
| Limonene (Dipentene No, 122, Hercules Inc.) | 0.1% | 0.1% | 0.1% | 0.1% |
| Citral | 0.1% | 0.1% | 0.1% | 0.1% |
| Preservative | q.s. | q.s. | q.s. | q.s. |
| Dyes | q.s. | q.s. | q.s. | q.s. |

### Recipes 8a, 8b, 8c and 8d: Micellar Water for Make-Up Removal

| | 8a | 8b | 8c | 8d |
|---|---|---|---|---|
| Water | to 100.0% | to 100.0% | to 100.0% | to 100.0% |
| Perfume | 0.1% | 0.1% | 0.1% | 0.1% |
| Composition from Example 1 | 2.0% | | | |
| Composition from Example 3 | | 2.0% | | |
| Composition from Example 4 | | | 2.0% | |
| Composition from Example 5 | | | | 2.0% |
| Capryl/Capramidopropyl Betaine (TEGO ® Betain | 1.3% | 1.3% | 1.3% | 1.3% |
| 810, Evonik, 38%) | | | | |
| Polyglyceryl-6 Caprylate; Polyglyceryl-3 Cocoate; | 1.0% | 1.0% | 1.0% | 1.0% |
| Polyglyceryl-4 Caprate; Polyglyceryl-6 Ricinoleate | | | | |
| (TEGO ® Solve 61, Evonik) | | | | |
| Betaine (TEGO ® Natural Betaine, Evonik) | 2.0% | 2.0% | 2.0% | 2.0% |
| Glycerol (Glycerol EP, vegetable, Spiga Nord) | 1.0% | 1.0% | 1.0% | 1.0% |
| Preservative | q.s. | q.s. | q.s. | q.s. |

### Recipes 9a, 9b, 9c and 9d: Solution for Wet Wipes

| Recipe | 9a | 9b | 9c | 9d |
|---|---|---|---|---|
| Composition from Example 1 | 3.5% | | | |
| Composition from Example 3 | | 3.5% | | |
| Composition from Example 4 | | | 3.5% | |
| Composition from Example 5 | | | | 3.5% |
| Aloe Barbadensis Leaf Extract (Aloe-Con UP 40, | 0.2% | 0.2% | 0.2% | 0.2% |
| Florida Food Products Inc.) | | | | |
| Isopropyl Myristate (TEGOSOFT ® M, Evonik) | 0.2% | 0.2% | 0.2% | 0.2% |
| Disodium Cocoamphodiacetate (REWOTERIC ® | 1.5% | 1.5% | 1.5% | 1.5% |
| AM 2 C NM, Evonik, 39%) | | | | |
| Perfume | 0.2% | 0.2% | 0.2% | 0.2% |
| Propylene Glycol (Euxyl K 320, Schülke & Mayr | 2.5% | 2.5% | 2.5% | 2.5% |
| GmbH) | | | | |
| Hydrolyzed Silk (Crosilk 10000, Croda Inc.) | 0.2% | 0.2% | 0.2% | 0.2% |
| Caprylyl/Capryl Glucoside (Plantacare 810 UP, | 1.0% | 1.0% | 1.0% | 1.0% |
| BASF) | | | | |
| Water | to 100.0% | to 100.0% | to 100.0% | to 100.0% |
| Citric Acid | to pH 5.0 | to pH 5.0 | to pH 5.0 | to pH 5.0 |
| Phenoxyethanol (S&M Phenoxyethanol, Schülke & | 0.5% | 0.5% | 0.5% | 0.5% |
| Mayr GmbH) | | | | |
| Dehydroacetic Acid (Unisept DHA (Universal | 0.1% | 0.1% | 0.1% | 0.1% |
| Preserv-A-Chem, Inc.) | | | | |
| Sodium Benzoate (Euxyl K 712, Schülke & Mayr | 0.4% | 0.4% | 0.4% | 0.4% |
| GmbH) | | | | |
| Salicylic Acid (Salicylic acid nat,; Evonik Dr. | 0.5% | 0.5% | 0.5% | 0.5% |
| Straetmans GmbH) | | | | |

### Recipes 10a, 10b, 10c and 10d: Antiperspirant Deodorant

| | | 10a | 10b | 10c | 10d |
|---|---|---|---|---|---|
| Phase | Composition from Example 1 | 4.0% | | | |
| A | Composition from Example 3 | | 4.0% | | |
| | Composition from Example 4 | | | 4.0% | |
| | Composition from Example 5 | | | | 4.0% |
| | Dicaprylyl Ether (Cetiol OE, BASF) | 0.3% | 0.3% | 0.3% | 0.3% |
| | Geraniol (Nerol 800, International Flavors | 0.1% | 0.1% | 0.1% | 0.1% |
| | & Fragrances Inc.) | | | | |
| | Linalool (Lipofresh, Lipo Chemicals, Inc.) | 0.1% | 0.1% | 0.1% | 0.1% |
| | Perfume | 0.1% | 0.1% | 0.1% | 0.1% |
| Phase | Propylene Glycol (Euxyl K 320, Schülke & | 1.0% | 1.0% | 1.0% | 1.0% |
| B | Mayr GmbH) | | | | |
| | Butylene Glycol (Oxea Corparation) | 0.2% | 0.2% | 0.2% | 0.2% |
| | Water | 5.0% | 5.0% | 5.0% | 5.0% |
| | Palmitamidopropyltrimonium Chloride | 1.0% | 1.0% | 1.0% | 1.0% |
| | (VARISOFT ® PATC, Evonik) | | | | |
| Phase | Water | 50.0% | 50.0% | 50.0% | 50.0% |
| C | Hydroxethyl Ethylcellulose (Structure Cel | 0.8% | 0.8% | 0.8% | 0.8% |
| | 4400 E, AkzoNobel) | | | | |
| | Sodium Hydroxide (10% in water) | 0.3% | 0.3% | 0.3% | 0.3% |
| Phase | Aluminium Chlorohydrate (Locron L, | 15.0% | 15.0% | 15.0% | 15.0% |
| D | Clariant) | | | | |
| Phase | Preservative | q.s. | q.s. | q.s. | q.s. |
| E | Water | to 100.0% | to 100.0% | to 100.0% | to 100.0% |

### Recipes 11a, 11b, 11c and 11d: Mouthwash

| | 11a | 11b | 11c | 11d |
|---|---|---|---|---|
| Composition from Example 1 | 0.4% | | | |
| Composition from Example 3 | | 0.4% | | |
| Composition from Example 4 | | | 0.4% | |
| Composition from Example 5 | | | | 0.4% |
| Glycolipids (Rheance One, Evonik) | 0.2% | 0.2% | 0.2% | 0.2% |
| Flavor | 0.2% | 0.2% | 0.2% | 0.2% |
| Water | to 100.0% | to 100.0% | to 100.0% | to 100.0% |
| Sorbitol (Karion FP Liquid, Merck) | 3.0% | 3.0% | 3.0% | 3.0% |
| Preservative | q.s. | q.s. | q.s. | q.s. |
| Dyes | q.s. | q.s. | q.s. | q.s. |

### Recipes 12a, 12b, 12c and 12d: Toothpaste

| | | 12a | 12b | 12c | 12d |
|---|---|---|---|---|---|
| A | Sorbitol (Karion FP Liquid, Merck) | 50.0% | 50.0% | 50.0% | 50.0% |
| | Water | to 100.0% | to 100.0% | to 100.0% | to 100.0% |
| | Sodium Carboxymethylcellulose (Blanose | 1.2% | 1.2% | 1.2% | 1.2% |
| | 7MXF, Ashland) | | | | |
| B | Sodium Saccharine (Sigma Aldrich) | 0.1% | 0.1% | 0.1% | 0.1% |
| | Sodium Fluoride (Sigma Aldrich) | 0.1% | 0.1% | 0.1% | 0.1% |
| C | Titanium Dioxide (Caesar & Loretz) | 0.4% | 0.4% | 0.4% | 0.4% |
| | Hydrated Silica (Zeodent ® 113, Evonik) | 14.0% | 14.0% | 14.0% | 14.0% |
| | Hydrated Silica (Zeodent ® 165, Evonik) | 8.0% | 8.0% | 8.0% | 8.0% |
| D | Flavor oil | 1.0% | 1.0% | 1.0% | 1.0% |
| E | Glyceryl Caprylate (dermosoft ® GMCY, | 0.3% | 0.3% | 0.3% | 0.3% |
| | Evonik) | | | | |
| | Composition from Example 1 | 3.5% | | | |
| | Composition from Example 3 | | 3.5% | | |
| | Composition from Example 4 | | | 3.5% | |
| | Composition from Example 5 | | | | 3.5% |

### Recipes 13a, 13b, 13c and 13d: Kitchen Cleaning Spray

| Ingredient | 13a | 13b | 13c | 13d |
|---|---|---|---|---|
| REWOTERIC ® AM V | 0.5% | 0.5% | 0.5% | 0.5% |
| Composition from Example 1 | 1.0% | | | |
| Composition from Example 3 | | 1.0% | | |
| Composition from Example 4 | | | 1.0% | |
| Composition from Example 5 | | | | 1.0% |
| TOMAKLEEN ® G-14 | 2.0% | 2.0% | 2.0% | 2.0% |
| Water (Dye, Perfume) | 89.5% | 89.5% | 89.5% | 89.5% |
| Chelating Agent, GLDA | 3.0% | 3.0% | 3.0% | 3.0% |
| Triethanolamine | 4.0% | 4.0% | 4.0% | 4.0% |

### Recipes 14a, 14b, 14c and 14d: Extra Mild Dish Wash Foam

| Ingredient | 14a | 14b | 14c | 14d |
|---|---|---|---|---|
| C10-16 Alkylpolyglucoside, 50% | 10.0% | 10.0% | 10.0% | 10.0% |
| Xanthan Gum | 0.1% | 0.1% | 0.1% | 0.1% |
| Glycerin | 2.0% | 2.0% | 2.0% | 2.0% |
| Water | 76.9% | 76.9% | 76.9% | 76.9% |
| Composition from Example 1 | 1.0% | | | |
| Composition from Example 3 | | 1.0% | | |
| Composition from Example 4 | | | 1.0% | |
| Composition from Example 5 | | | | 1.0% |
| REWOFERM ® SL ONE | 2.0% | 2.0% | 2.0% | 2.0% |
| REWOPOL ® SB CS 50 | 5.0% | 5.0% | 5.0% | 5.0% |
| TEGO ® Betain C 60 | 3.0% | 3.0% | 3.0% | 3.0% |
| Preservative | qs. | qs. | qs. | qs. |

### Recipes 15a, 15b, 15c and 15d: Automatic Rinse Aid for Direct Use 1

| Ingredient | 15a | 15b | 15c | 15d |
|---|---|---|---|---|
| TEGOTENS ® EC 11 | 5.0% | 5.0% | 5.0% | 5.0% |
| Composition from Example 1 | 5.0% | | | |
| Composition from Example 3 | | 5.0% | | |
| Composition from Example 4 | | | 5.0% | |
| Composition from Example 5 | | | | 5.0% |
| Citric acid monohydrate | 5.0% | 5.0% | 5.0% | 5.0% |
| Water (Dye, Perfume) | 85.0% | 85.0% | 85.0% | 85.0% |

### Recipes 16a, 16b, 16c and 16d: Automatic Rinse Aid for Direct Use 2

| Ingredient | 16a | 16b | 16c | 16d |
|---|---|---|---|---|
| Citric acid monohydrate | 5.0% | 5.0% | 5.0% | 5.0% |
| Sodium Cumene Sulfonate, 92% | 1.5% | 1.5% | 1.5% | 1.5% |
| Water (Dye, Perfume) | 83.5% | 83.5% | 83.5% | 83.5% |
| TEGOTENS ® EC 11 | 6.0% | 6.0% | 6.0% | 6.0% |
| Composition from Example 1 | 4.0% | | | |
| Composition from Example 3 | | 4.0% | | |
| Composition from Example 4 | | | 4.0% | |
| Composition from Example 5 | | | | 4.0% |

### Recipes 17a, 17b, 17c and 17d: Automatic Rinse Aid for Direct Use 3

| Ingredient | 17a | 17b | 17c | 17d |
|---|---|---|---|---|
| Citric acid monohydrate | 5.0% | 5.0% | 5.0% | 5.0% |
| Sodium Cumene Sulfonate, 92% | 1.5% | 1.5% | 1.5% | 1.5% |
| Water (Dye, Perfume) | 83.5% | 83.5% | 83.5% | 83.5% |
| TEGOTENS ® EC 11 | 7.0% | 7.0% | 7.0% | 7.0% |
| Composition from Example 1 | 3.0% | | | |
| Composition from Example 3 | | 3.0% | | |
| Composition from Example 4 | | | 3.0% | |
| Composition from Example 5 | | | | 3.0% |

Recipes 18a, 18b, 18c and 18d: Glass Cleaner with Optimized Antifogging Efficiency

| Ingredient | 18a | 18b | 18c | 18d |
|---|---|---|---|---|
| REWOPOL ® TS 35 | 1.0% | 1.0% | 1.0% | 1.0% |
| Composition from Example 1 | 0.2% | | | |
| Composition from Example 3 | | 0.2% | | |
| Composition from Example 4 | | | 0.2% | |
| Composition from Example 5 | | | | 0.2% |
| Isopropanol | 15.0% | 15.0% | 15.0% | 15.0% |
| Chelating Agent MGDA | 2.0% | 2.0% | 2.0% | 2.0% |
| Water (Dye, Perfume) | 81.8% | 81.8% | 81.8% | 81.8% |

### Recipes 19a, 19b, 19c and 19d: Oven Cleanser for Smoking Chamber

| Ingredient | 19a | 19b | 19c | 19d |
|---|---|---|---|---|
| Water | 77.6% | 77.6% | 77.6% | 77.6% |
| Trilon A liquid | 8.0% | 8.0% | 8.0% | 8.0% |
| Sodium carbonate | 6.0% | 6.0% | 6.0% | 6.0% |
| REWOTERIC ® AM KSF 40 | 5.0% | 5.0% | 5.0% | 5.0% |
| Laureth-6 | 1.2% | 1.2% | 1.2% | 1.2% |
| Composition from Example 1 | 1.2% | | | |
| Composition from Example 3 | | 1.2% | | |
| Composition from Example 4 | | | 1.2% | |
| Composition from Example 5 | | | | 1.2% |
| DOWANOL DPnB | 1.0% | 1.0% | 1.0% | 1.0% |

### Recipes 20a, 20b, 20c and 20d: All Purpose Cleanser (Microemulsion)

| Ingredient | 20a | 20b | 20c | 20d |
|---|---|---|---|---|
| Water | 73.0% | 73.0% | 73.0% | 73.0% |
| TEGOTENS ® AM VSF | 6.0% | 6.0% | 6.0% | 6.0% |
| Composition from Example 1 | 2.0% | | | |
| Composition from Example 3 | | 2.0% | | |
| Composition from Example 4 | | | 2.0% | |
| Composition from Example 5 | | | | 2.0% |
| REWOPOL ® D 510 NC | 5.0% | 5.0% | 5.0% | 5.0% |
| Undeceth-6 | 3.0% | 3.0% | 3.0% | 3.0% |
| Potassium hydroxide, 50% | 5.0% | 5.0% | 5.0% | 5.0% |
| Chelating Agent MGDA | 6.0% | 6.0% | 6.0% | 6.0% |

### Recipes 21a, 21b, 21c and 21d: Low Foaming Hard Surface Degreaser

| Ingredient | 21a | 21b | 21c | 21d |
|---|---|---|---|---|
| C9-11 FATTY ALCOHOL | 2.0% | 2.0% | 2.0% | 2.0% |
| ETHOXYLATE - 8 MOLES | | | | |
| OF EO | | | | |
| Composition from Example 1 | 1.0% | | | |
| Composition from Example 3 | | 1.0% | | |
| Composition from Example 4 | | | 1.0% | |
| Composition from Example 5 | | | | 1.0% |
| TEGOTENS ® G 826 C | 4.0% | 4.0% | 4.0% | 4.0% |
| Chelating Agent MGDA | 7.5% | 7.5% | 7.5% | 7.5% |
| Water (Dye, Perfume) | 85.5% | 85.5% | 85.5% | 85.5% |

### Recipes 22a, 22b, 22c and 22d: Low Foaming Hard Surface Degreaser (101630-23)

| Ingredient | 22a | 22b | 22c | 22d |
|---|---|---|---|---|
| C9-11 FATTY ALCOHOL | 2.0% | 2.0% | 2.0% | 2.0% |
| ETHOXYLATE - 8 MOLES | | | | |
| OF EO | | | | |
| Composition from Example 1 | 1.0% | | | |
| Composition from Example 3 | | 1.0% | | |
| Composition from Example 4 | | | 1.0% | |
| Composition from Example 5 | | | | 1.0% |
| TEGOTENS ® G 826 C | 4.0% | 4.0% | 4.0% | 4.0% |
| Chelating Agent MGDA | 7.5% | 7.5% | 7.5% | 7.5% |
| Water (Dye, Perfume) | 85.5% | 85.5% | 85.5% | 85.5% |

### Recipes 23a, 23b, 23c and 23d: Foaming Hard Surface Degreaser 1

| Ingredient | 23a | 23b | 23c | 23d |
|---|---|---|---|---|
| C9-11 FATTY ALCOHOL | 2.0% | 2.0% | 2.0% | 2.0% |
| ETHOXYLATE - 8 MOLES | | | | |
| OF EO | | | | |
| TEGOTENS ® AM VSF | 6.0% | 6.0% | 6.0% | 6.0% |
| Composition from Example 1 | 1.0% | | | |
| Composition from Example 3 | | 1.0% | | |
| Composition from Example 4 | | | 1.0% | |
| Composition from Example 5 | | | | 1.0% |
| Chelating Agent MGDA | 7.5% | 7.5% | 7.5% | 7.5% |
| Water (Dye, Perfume) | 83.5% | 83.5% | 83.5% | 83.5% |

### Recipes 24a, 24b, 24c, and 24d: Foaming Hard Surface Degreaser 2

| Ingredient | 24a | 24b | 24c | 24d |
|---|---|---|---|---|
| C9-11 FATTY ALCOHOL | 3.0% | 3.0% | 3.0% | 3.0% |
| ETHOXYLATE - 8 MOLES | | | | |
| OF EO | | | | |
| REWOTERIC ® AM KSF 40 | 2.5% | 2.5% | 2.5% | 2.5% |
| Composition from Example 1 | 1.0% | | | |
| Composition from Example 3 | | 1.0% | | |
| Composition from Example 4 | | | 1.0% | |
| Composition from Example 5 | | | | 1.0% |
| Chelating Agent MGDA | 10.0% | 10.0% | 10.0% | 10.0% |
| Water (Dye, Perfume) | 83.5% | 83.5% | 83.5% | 83.5% |

### Recipes 25a, 25b, 25c and 25d: Low Foaming Hard Surface Degreaser

| Ingredient | 25a | 25b | 25c | 25d |
|---|---|---|---|---|
| Composition from Example 1 | 1.0% | | | |
| Composition from Example 3 | | 1.0% | | |
| Composition from Example 4 | | | 1.0% | |
| Composition from Example 5 | | | | 1.0% |
| C8-10 Alkylpolyglucosid | 4.0% | 4.0% | 4.0% | 4.0% |
| Sodium C13-17 Alkane Sulfonate, | 2.0% | 2.0% | 2.0% | 2.0% |
| 30% | | | | |
| Chelating Agent MGDA | 7.5% | 7.5% | 7.5% | 7.5% |
| Water (Dye, Perfume) | 85.5% | 85.5% | 85.5% | 85.5% |

Recipes 26a, 26b, 26c and 26d: Hard Surface Degreaser from Renewable Surfactants

| Ingredient | 26a | 26b | 26c | 26d |
|---|---|---|---|---|
| Water | 89.3% | 89.3% | 89.3% | 89.3% |
| Chelating Agent MGDA | 4.0% | 4.0% | 4.0% | 4.0% |
| Sodium carbonate | 3.0% | 3.0% | 3.0% | 3.0% |
| TEGOTENS ® AM VSF | 2.4% | 2.4% | 2.4% | 2.4% |
| Laureth-6 | 0.6% | 0.6% | 0.6% | 0.6% |
| Composition from Example 1 | 0.4% | | | |
| Composition from Example 3 | | 0.4% | | |
| Composition from Example 4 | | | 0.4% | |
| Composition from Example 5 | | | | 0.4% |
| TEGOR Polish Additiv Q 70 | 0.3% | 0.3% | 0.3% | 0.3% |

### Recipes 27a, 27b, 27c and 27d: Highly Efficient Floor Cleanser

| Ingredient | 27a | 27b | 27c | 27d |
|---|---|---|---|---|
| Water | 89.3% | 89.3% | 89.3% | 89.3% |
| Chelating Agent MGDA | 4.0% | 4.0% | 4.0% | 4.0% |
| Sodium carbonate | 3.0% | 3.0% | 3.0% | 3.0% |
| TEGOTENS ® AM VSF | 2.4% | 2.4% | 2.4% | 2.4% |
| Laureth-6 | 0.6% | 0.6% | 0.6% | 0.6% |
| Composition from Example 1 | 0.4% | | | |
| Composition from Example 3 | | 0.4% | | |
| Composition from Example 4 | | | 0.4% | |
| Composition from Example 5 | | | | 0.4% |
| TEGOR Polish Additiv Q 70 | 0.3% | 0.3% | 0.3% | 0.3% |

### Recipes 28a, 28b, 28c and 28d: Super Natural Wash Lotion for Textile Face Masks

| Ingredient | 28a | 28b | 28c | 28d |
|---|---|---|---|---|
| Xanthan Gum | 0.1% | 0.1% | 0.1% | 0.1% |
| Glycerin | 2.0% | 2.0% | 2.0% | 2.0% |
| Water | 76.9% | 76.9% | 76.9% | 76.9% |
| Composition from Example 1 | 1.0% | | | |
| Composition from Example 3 | | 1.0% | | |
| Composition from Example 4 | | | 1.0% | |
| Composition from Example 5 | | | | 1.0% |
| REWOFERM ® SL ONE | 2.0% | 2.0% | 2.0% | 2.0% |
| REWOPOL ® SB CS 50 | 5.0% | 5.0% | 5.0% | 5.0% |
| C8-10 Alkylpolyglucosid | 10.0% | 10.0% | 10.0% | 10.0% |
| TEGO ® Betain C 60 | 3.0% | 3.0% | 3.0% | 3.0% |
| Preservative | qs. | qs. | qs. | qs. |

### Recipes 29a, 29b, 29c and 29d: Highly Effective Presoaker

| Ingredient | 29a | 29b | 29c | 29d |
|---|---|---|---|---|
| Water | 77.5% | 77.5% | 77.5% | 77.5% |
| Chelating Agent MGDA | 8.0% | 8.0% | 8.0% | 8.0% |
| Sodium metasilicate | 2.0% | 2.0% | 2.0% | 2.0% |
| Sodium gluconate | 2.0% | 2.0% | 2.0% | 2.0% |
| Sodium carbonate | 2.0% | 2.0% | 2.0% | 2.0% |
| TEGOTENS ® AM VSF | 6.5% | 6.5% | 6.5% | 6.5% |
| Undeceth-6 | 0.6% | 0.6% | 0.6% | 0.6% |
| Composition from Example 1 | 0.4% | | | |
| Composition from Example 3 | | 0.4% | | |
| Composition from Example 4 | | | 0.4% | |
| Composition from Example 5 | | | | 0.4% |
| DOWANOL DPnB | 1.0% | 1.0% | 1.0% | 1.0% |

### Recipes 30a, 30b, 30c and 30d: Presoaker (Basic Formula)

| Ingredient | 30a | 30b | 30c | 30d |
|---|---|---|---|---|
| Water | 82.6% | 82.6% | 82.6% | 82.6% |
| Trilon A 92 | 3.0% | 3.0% | 3.0% | 3.0% |
| Sodium metasilicate | 2.0% | 2.0% | 2.0% | 2.0% |
| Sodium carbonate | 2.0% | 2.0% | 2.0% | 2.0% |
| Bayhibit AM | 1.0% | 1.0% | 1.0% | 1.0% |
| REWOTERIC ® AM KSF 40 | 4.0% | 4.0% | 4.0% | 4.0% |
| C9-11 FATTY ALCOHOL | 1.2% | 1.2% | 1.2% | 1.2% |
| ETHOXYLATE - 8 MOLES | | | | |
| OF EO | | | | |
| Composition from Example 1 | 1.2% | | | |
| Composition from Example 3 | | 1.2% | | |
| Composition from Example 4 | | | 1.2% | |
| Composition from Example 5 | | | | 1.2% |
| DOWANOL DPnB | 3.0% | 3.0% | 3.0% | 3.0% |

### Recipes 31a, 31b, 31c and 31d: Good Dispersing Presoaker

| Ingredient | 31a | 31b | 31c | 31d |
|---|---|---|---|---|
| Water | 80.0% | 80.0% | 80.0% | 80.0% |
| Trilon A 92 | 3.0% | 3.0% | 3.0% | 3.0% |
| Sodium metasilicate | 2.0% | 2.0% | 2.0% | 2.0% |
| Sodium carbonate | 2.0% | 2.0% | 2.0% | 2.0% |
| Sequion 10 Na 430 | 2.0% | 2.0% | 2.0% | 2.0% |
| TEGOTENS ® 475 | 5.0% | 5.0% | 5.0% | 5.0% |
| C9-11 FATTY ALCOHOL | 1.5% | 1.5% | 1.5% | 1.5% |
| ETHOXYLATE - 7 MOLES | | | | |
| OF EO | | | | |
| Composition from Example 1 | 1.5% | | | |
| Composition from Example 3 | | 1.5% | | |
| Composition from Example 4 | | | 1.5% | |
| Composition from Example 5 | | | | 1.5% |
| DOWANOL PnB | 3.0% | 3.0% | 3.0% | 3.0% |

### Recipes 32a, 32b, 32c and 32d: Cost Efficient Presoaker

| Ingredient | 32a | 32b | 32c | 32d |
|---|---|---|---|---|
| Water | 80.5% | 80.5% | 80.5% | 80.5% |
| Chelating Agent MDGA | 8.0% | 8.0% | 8.0% | 8.0% |
| Sodium carbonate | 6.0% | 6.0% | 6.0% | 6.0% |
| TEGOTENS ® AM VSF | 3.5% | 3.5% | 3.5% | 3.5% |
| Laureth-6 | 0.6% | 0.6% | 0.6% | 0.6% |
| Composition from Example 1 | 0.4% | | | |
| Composition from Example 3 | | 0.4% | | |
| Composition from Example 4 | | | 0.4% | |
| Composition from Example 5 | | | | 0.4% |
| DOWANOL DPnB | 1.0% | 1.0% | 1.0% | 1.0% |

### Recipes 33a, 33b, 33c and 33d: Rinse Aid to Minimize Remaining Water

| Ingredient | 33a | 33b | 33c | 33d |
|---|---|---|---|---|
| REWOPAL ® MPG 40 | 6.0% | 6.0% | 6.0% | 6.0% |
| Butyl Cellusolve (BG) | 14.0% | 14.0% | 14.0% | 14.0% |
| Composition from Example 1 | 3.0% | | | |
| Composition from Example 3 | | 3.0% | | |
| Composition from Example 4 | | | 3.0% | |
| Composition from Example 5 | | | | 3.0% |
| REWOQUAT ® CR 3099 | 10.0% | 10.0% | 10.0% | 10.0% |
| TEGOSOFT ® OP | 5.0% | 5.0% | 5.0% | 5.0% |
| Water | 62.0% | 62.0% | 62.0% | 62.0% |

5 Recipes 34a, 34b, 34c and 34d: Rinse Aid with Optimal Oil Content

| Ingredient | 34a | 34b | 34c | 34d |
|---|---|---|---|---|
| DOWANOL DPnB | 8.6% | 8.6% | 8.6% | 8.6% |
| REWOPAL ® MPG 40 | 6.2% | 6.2% | 6.2% | 6.2% |
| REWOQUAT ® CR 3099 | 10.0% | 10.0% | 10.0% | 10.0% |
| REWOCARE ® DOC | 4.0% | 4.0% | 4.0% | 4.0% |
| TEGOSOFT ® OP | 3.0% | 3.0% | 3.0% | 3.0% |
| Composition from Example 1 | 1.0% | | | |
| Composition from Example 3 | | 1.0% | | |
| Composition from Example 4 | | | 1.0% | |
| Composition from Example 5 | | | | 1.0% |
| Water | 66.8% | 66.8% | 66.8% | 66.8% |
| TEGOPREN ® 6923 | 0.4% | 0.4% | 0.4% | 0.4% |

### Recipes 35a, 35b, 35c and 35d: Low Foaming all Purpose Cleaner

| Ingredient | 35a | 35b | 35c | 35d |
|---|---|---|---|---|
| Water | 82.0% | 82.0% | 82.0% | 82.0% |
| Trilon A 92 | 3.0% | 3.0% | 3.0% | 3.0% |
| Sodium metasilicate | 2.0% | 2.0% | 2.0% | 2.0% |
| Sodium carbonate | 2.0% | 2.0% | 2.0% | 2.0% |
| Sequion 10 Na 430 | 2.0% | 2.0% | 2.0% | 2.0% |
| TEGOTENS ® 475 | 5.0% | 5.0% | 5.0% | 5.0% |
| C9-11 FATTY ALCOHOL | 1.5% | 1.5% | 1.5% | 1.5% |
| ETHOXYLATE - 7 MOLES | | | | |
| OF EO | | | | |
| Composition from Example 1 | 1.5% | | | |
| Composition from Example 3 | | 1.5% | | |
| Composition from Example 4 | | | 1.5% | |
| Composition from Example 5 | | | | 1.5% |
| DOWANOL PnB | 1.0% | 1.0% | 1.0% | 1.0% |

### Recipes 36a, 36b, 36c and 36d: Low Foaming Alkaline Cleanser

| Ingredient | 36a | 36b | 36c | 36d |
|---|---|---|---|---|
| Water | 85.5% | 85.5% | 85.5% | 85.5% |
| Chelating Agent MDGA | 4.0% | 4.0% | 4.0% | 4.0% |
| Sodium carbonate | 6.0% | 6.0% | 6.0% | 6.0% |
| TEGOTENS ® AM VSF | 3.5% | 3.5% | 3.5% | 3.5% |
| Laureth-6 | 0.6% | 0.6% | 0.6% | 0.6% |
| Composition from Example 1 | 0.4% | | | |
| Composition from Example 3 | | 0.4% | | |
| Composition from Example 4 | | | 0.4% | |
| Composition from Example 5 | | | | 0.4% |

### Recipes 37a, 37b, 37c and 37d: Low Foaming Alkaline Cleaner

| Ingredient | 37a | 37b | 37c | 37d |
|---|---|---|---|---|
| Water | 85.35% | 85.35% | 85.35% | 85.35% |
| TOMAKLEEN G-14 | 0.75% | 0.75% | 0.75% | 0.75% |
| TEGOTENS ® AM VSF | 3.5% | 3.5% | 3.5% | 3.5% |
| Composition from Example 1 | 0.4% | | | |
| Composition from Example 3 | | 0.4% | | |
| Composition from Example 4 | | | 0.4% | |
| Composition from Example 5 | | | | 0.4% |
| Sodium carbonate | 6.0% | 6.0% | 6.0% | 6.0% |
| MGDA-Na3 (40%) | 4.0% | 4.0% | 4.0% | 4.0% |

### Recipes 38a, 38b, 38c and 38d: Alkaline Cleanser (Automatic Wash)

| Ingredient | 38a | 38b | 38c | 38d |
|---|---|---|---|---|
| Water | 59.0% | 59.0% | 59.0% | 59.0% |
| Tetra Potassium Pyrophosphate | 15.0% | 15.0% | 15.0% | 15.0% |
| (TKPP) | | | | |
| Trilon A liquid | 5.0% | 5.0% | 5.0% | 5.0% |
| KOH, 45% | 4.0% | 4.0% | 4.0% | 4.0% |
| Sequion 10 Na 430 | 10.0% | 10.0% | 10.0% | 10.0% |
| Sodium metasilicate | 6.0% | 6.0% | 6.0% | 6.0% |
| Composition from Example 1 | 1.0% | | | |
| Composition from Example 3 | | 1.0% | | |
| Composition from Example 4 | | | 1.0% | |
| Composition from Example 5 | | | | 1.0% |

### Recipes 39a, 39b, 39c and 39d: Metal Cleaner

| Ingredient | 39a | 39b | 39c | 39d |
|---|---|---|---|---|
| Water | 82.0% | 82.0% | 82.0% | 82.0% |
| TOMAKLEEN ® G-14 | 12.0% | 12.0% | 12.0% | 12.0% |
| REWOTERIC ® AM V | 1.5% | 1.5% | 1.5% | 1.5% |
| Composition from Example 1 | 3.0% | | | |
| Composition from Example 3 | | 3.0% | | |
| Composition from Example 4 | | | 3.0% | |
| Composition from Example 5 | | | | 3.0% |
| Triethanolamine | 1.5% | 1.5% | 1.5% | 1.5% |

### Recipes 40a, 40b, 40c and 40d: Metal Cleanser

| Ingredient | 40a | 40b | 40c | 40d |
|---|---|---|---|---|
| Water (Dye, Perfume) | 82.0% | 82.0% | 82.0% | 82.0% |
| Triethanolamine | 1.5% | 1.5% | 1.5% | 1.5% |
| DOWANOL PnP | 6.0% | 6.0% | 6.0% | 6.0% |
| REWOTERIC ® AM V | 1.5% | 1.5% | 1.5% | 1.5% |
| Composition from Example 1 | 3.0% | | | |
| Composition from Example 3 | | 3.0% | | |
| Composition from Example 4 | | | 3.0% | |
| Composition from Example 5 | | | | 3.0% |
| C8-10 Fatty alcohol•5 EO | 6.0% | 6.0% | 6.0% | 6.0% |

### Recipes 41a, 41b, 41c and 41d: Cost Efficient Facade Cleanser

| Ingredient | 41a | 41b | 41c | 41d |
|---|---|---|---|---|
| Water | 86.6% | 86.6% | 86.6% | 86.6% |
| Chelating Agent, MGDA | 4.0% | 4.0% | 4.0% | 4.0% |
| Sodium carbonate | 6.0% | 6.0% | 6.0% | 6.0% |
| TEGOTENS ® AM VSF | 2.4% | 2.4% | 2.4% | 2.4% |
| Laureth-6 | 0.6% | 0.6% | 0.6% | 0.6% |
| Composition from Example 1 | 0.4% | | | |
| Composition from Example 3 | | 0.4% | | |
| Composition from Example 4 | | | 0.4% | |
| Composition from Example 5 | | | | 0.4% |

1. A mixture of n-nonanoic esters of xylitol, sorbitol or erythritol, comprising: at least two of the esters which differ with regard to at least one esterification position of at least one nonanoyl radical in the xylitol, sorbitol or erythritol, with the proviso that n-nonanoic esters of erythritol with an average level of esterification of greater than 3.2 are excluded.

2. The mixture of n-nonanoic esters according to claim 1, wherein it comprises at least two regioisomers of the mono-n-nonanoic ester.

3. The mixture of n-nonanoic esters according to claim 1, wherein it comprises mono-n-nonanoic ester and di-n-nonanoic ester.

4. The mixture of n-nonanoic esters according to claim 1, wherein it has an average level of esterification of 1.0 to 4.0.

5. A mixed composition comprising the mixture of n-nonanoic esters according to claim 1, wherein said composition comprises less than 25% by weight of free n-nonanoic acid, where the percentages by weight are based on the sum total of all n-nonanoic esters of xylitol, sorbitol and erythritol and n-nonanoic acid.

6. The mixed composition according to claim 5, wherein said composition comprises 0.05% by weight to 40% by weight of free xylitol, sorbitol and/or erythritol, where the percentages by weight are based on the sum total of all n-nonanoic esters of xylitol, sorbitol and erythritol and all xylitol, sorbitol and erythritol.

7. The mixed composition according to claim 5, wherein said composition comprises 0.1% by weight to 60% by weight of at least one solvent.

8. A process for enzymatic preparation of a mixture of n-nonanoic esters of xylitol, sorbitol or erythritol according to claim 1, comprising:
A) providing xylitol, sorbitol or erythritol and at least one n-nonanoyl group donor,
B) reacting xylitol, sorbitol or erythritol with the at least one n-nonanoyl group donor in the presence of a lipase at a temperature of 75° C. to 110° C. to give an n-nonanoic ester of xylitol, sorbitol or erythritol, and optionally
C) purifying the n-nonanoic ester of xylitol, sorbitol or erythritol.

9. The process according to claim 8, wherein A) comprises blending the xylitol, sorbitol or erythritol with the at least one n-nonanoyl group donor for at least ten minutes.

10. The process according to claim 8, wherein the xylitol, sorbitol or erythritol with the at least one n-nonanoyl group donor accounts for at least 80% by weight based on the overall reaction mixture at the start of B).

11. The process according to claim 8, wherein the lipase is at least one selected from the group comprising the lipase from Thermomyces lanuginosus (accession number 059952), lipases A and B (accession number P41365) from Candida antarctica, and the lipase from Mucor miehei (accession number P19515), the lipase from Humicola sp. (accession number OS9952), the lipase from Rhizomucor javanicus (accession number S32492), the lipase from Rhizopus oryzae (accession number P61872), the lipases from Candida rugosa (accession number P20261, P32946, P32947, P3294 and P32949), the lipase from Rhizopus niveus (accession number P61871), the lipase from Penicillium camemberti (accession number P25234), the lipases from Aspergillus niger (ABG73613, ABG73614 and ABG37906), the lipase from Penicillium cyclopium (accession number P61869), and their respective at least 60% homologues at the amino acid level.

12. The process according to claim 8, wherein B) is conducted at a pressure of less than 1 bar.

13. The process according to claim 8, wherein, in A), as well as the xylitol, sorbitol or erythritol, at least one other sugar or sugar alcohol is provided, selected from the group consisting of agarose, allitol, allulose, altritol, amylopectin, amylose, arabinitol, arabinose, cellobiose, cellulose, chitin, cyclodextrins, deoxyribose, dextrans, erythritol, fructans, fructose, fucose, galactitol, galactose, glucitol, glucose, glycogen, hyaluronic acid, iditol, inulin, isomalt, isomaltulose, isomelizitose, lactitol, lactose, lactulose, maltitol, maltohexose, maltopentose, maltose, maltotetrose, maltotriose, maltulose, mannitol, mannose, melizitose, pectins, raffinose, rhamnose, ribitol, ribose, sucrose, sorbitol, sorbose, stachyose, starch, starch hydrolysate, threitol, trehalulose, umbelliferose, xylitol and xylose, and this also passes through the further process steps.

14. The n-nonanoic ester of xylitol, sorbitol or erythritol, obtainable by a process according to claim 8.

15. A method of making a product, comprising:
producing at least one selected from the group consisting of a viscosity regulator, active care ingredient, foam booster or solubilizer, antimicrobial, antistat, binder, corrosion inhibitor, dispersant, emulsifier, film former, humectant, opacifier, oral care agent, preservative, skincare agent, hydrophilic emollient, foam stabilizer and/or nonionic surfactant with at least one of the mixture of n-nonanoic esters of xylitol, sorbitol, or erythritol according to claim 1.

16. The mixture of n-nonanoic esters according to claim 1, wherein it comprises mono-n-nonanoic ester, di-n-nonanoic ester, and tri-n-nonanoic ester.

17. The mixture of n-nonanoic esters according to claim 1, wherein it has an average level of esterification of 1.0 to 3.0.

18. The mixture of n-nonanoic esters according to claim 1, wherein it has an average level of esterification of 1.1 to 2.7.

19. The mixture of n-nonanoic esters according to claim 1, wherein it has an average level of esterification of 1.3 to 2.6.
